(19) United States
(12) Patent Application Publication
(10) Pub. No.: US 2009/0131480 A1
(43) Pub. Date: May 21, 2009

(54) Dihydropyridine Compounds for Neurodegenerative Diseases and Dementia

(75) Inventors: Julian A. Gray, Inzlingen (DE); Yoshiharu Yamanishi, Ibaraki (JP); Julian A. Gray, Inzlingen (DE); Yoshiharu Yamanishi, Ibaraki (JP); Nobuyuki Mori, Ibaraki (JP); Scott Fields, Towaco, NJ (US)

Correspondence Address: VENABLE LLP, P.O. BOX 34385, WASHINGTON, DC, 20043-9998, US

(73) Assignee: EISAI Co., Ltd., Tokyo (JP)

(21) Appl. No.: 11/887,729

(22) PCT Filed: Apr. 4, 2006
(86) PCT No.: PCT/US06/12284

§ 371 Date: Jan. 14, 2009

Related U.S. Application Data
(60) Provisional application No. 60/667,665, filed on Apr. 4, 2005.
(60) Provisional application No. 60/689,519, filed on Jun. 13, 2005.

(51) Int. Cl. A61K 31/444; A61P 25/28

(52) U.S. Cl. 514/332

(57) ABSTRACT

The invention provides methods for treating and/or preventing cognitive impairments, dementia, or neurodegenerative diseases and disorders (e.g., Alzheimer's disease, Parkinson's disease, Huntington's disease, amyotrophic lateral sclerosis) in patients by administering therapeutically effective amounts of an AMPA receptor antagonist (e.g., 1,2-dihydropyridine compounds) and therapeutically effective amounts of nootropics (e.g., cholinesterase inhibitors) to patients. The invention also provides combinations, commercial packages, and pharmaceutical compositions comprising therapeutically effective amounts of AMPA receptor antagonists (e.g., 1,2-dihydropyridine compounds) and therapeutically effective amounts nootropics (e.g., cholinesterase inhibitors). The 1,2-dihydropyridine compound may be, for example, 3-(2-cyanophenyl)-5-(2-pyridyl)-1-phenyl-1,2-dihydropyridin-2-one. The cholinesterase inhibitor may be, for example, donepezil.

### RELATED APPLICATIONS

This application claims priority under 35 U.S.C. § 119 to U.S. Provisional Application No. 60/689,519 filed Jun. 13, 2005, and to U.S. Provisional Application No. 60/667,665 filed Apr. 4, 2005, the disclosures of which are incorporated by reference herein in their entirety.

### FIELD OF THE INVENTION

The invention provides pharmaceutical compositions comprising dihydropyridine compounds and methods for treating a variety of diseases and disorders using dihydropyridine compounds. The dihydropyridine compounds can optionally be used in conjunction with other drugs, such as cholinesterase inhibitors, for treating a variety of diseases and disorders.

### BACKGROUND OF THE INVENTION

Excitatory amino acid receptors are classified into two general types. Receptors that are directly coupled to the opening of cation channels in the cell membrane of the neurons are termed “ionotropic.” This type of receptor has been subdivided into at least three subtypes, which are defined by the depolarizing actions of the selective agonist N-methyl-D-aspartate (NMDA), α-amino-3-hydroxy-5-methylisoxazole-4-propionic acid (AMPA), and kainic acid. The second general type is the G-protein or second messenger-linked “metabotropic” excitatory amino acid receptor. This second type, when activated by the agonists quisqualate, ibotenate, or trans-1-aminocyclopentane-1,3-dicarboxylic acid, leads to enhanced phosphoinositide hydrolysis in the postsynaptic cell. Both types of receptors appear not only to mediate normal synaptic connections during development, but also change in the efficiency of synaptic transmission throughout life. Schoepp et al, Trends Pharm. Sci., 11:508 (1990); McDonald et al, Brain Res. Rev., 15:41 (1990).

Excessive excitation by neurotransmitters can cause the degeneration and death of neurons. It is believed that this degeneration is in part mediated by the excitotoxic actions of the excitatory amino acids, glutamate and aspartate, at the NMDA receptor, the AMPA receptor, and the kainate receptor. This excitotoxic action is responsible for the loss of neurons in cerebrovascular disorders such as cerebral ischemia or cerebral infarction resulting from a range of conditions, such as thromboembolic or haemorrhagic stroke, cerebral vasospasm, hypoglycaemia, cardiac arrest, status epilepticus, perinatal asphyxia, anoxia such as from near-drowning, pulmonary surgery and cerebral trauma as well as lathyrism, Alzheimer's, and Huntington's diseases. U.S. Pat. Nos. 6,765,006 and 5,843,945.

There is a need in the art for new compounds and new methods of treating diseases and disorders that may be mediated to any extent by NMDA receptors, AMPA receptors and/or kainate receptors. The invention is directed to this, as well as other, important ends.

### SUMMARY OF THE INVENTION

The invention provides methods for treatment and/or prophylaxis of dementia or cognitive impairments in a patient in need thereof by administering a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one). The invention also provides methods for delaying the onset of dementia or cognitive impairments by administering a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one). The combination of the 1,2-dihydropyridine compound and the cholinesterase inhibitor unexpectedly produces synergistic effects in the treatment and/or prophylaxis of dementia or cognitive impairments.

In other embodiments, the invention provides methods for treatment and/or prophylaxis of neurodegenerative diseases (e.g., Alzheimer's disease, Parkinson's disease, Huntington's disease, amyotrophic lateral sclerosis, and the like) in a patient in need thereof by administering a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one). The invention also provides methods for delaying the onset of neurodegenerative diseases (e.g., Alzheimer's disease, Parkinson's disease, Huntington's disease, amyotrophic lateral sclerosis, and the like) by administering a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one). The combination of the 1,2-dihydropyridine compound and the cholinesterase inhibitor unexpectedly produce synergistic effects in the treatment of neurodegenerative diseases.

In other embodiments, the invention provides pharmaceutical compositions comprising a therapeutically effective amount of: (i) at least one cholinesterase inhibitor (e.g., donepezil) and (ii) at least one 1,2-dihydropyridine compound (e.g., 3-(2-cyanophenyl)-5-(2-pyridyl)-1-phenyl-1,2-dihydropyridin-2-one).

In other embodiments, the invention provides combinations comprising an effective amount of: (i) at least one AMPA receptor antagonist and (ii) at least one nootropic. The combination may be separately administered (e.g., simultaneously, sequentially) to a patient to treat dementia or one or more behavioral disturbances caused by dementia. The combination may also be administered separately (e.g., simultaneously, sequentially) to a patient to treat Alzheimer's disease. The invention also provides commercial packages or kits comprising an effective amount of: (i) at least one AMA receptor antagonist; (ii) at least one nootropic; and (iii) instructions for simultaneous, separate or sequential use thereof in the treatment of dementia or behavioral disturbances observed with dementia. In one embodiment, the instructions are for the treatment of Alzheimer's disease. The invention also provides pharmaceutical compositions comprising an effective amount of: (i) at least one AMPA receptor antagonist; (ii) at least one nootropic; and (iii) a pharmaceutically acceptable excipient.

### BRIEF DESCRIPTION OF THE FIGURES

FIG. 1 shows the effect of Compound A (i.e., 3-(2-cyanophenyl)-5-(2-pyridyl)-1-phenyl-1,2-dihydropyridin-2-one) on dopamine, DOPAC, and HVA content in mice striatum.

FIG. 2 shows the effects of donepezil on dopamine turnover in mice striatum.

FIG. 3A-E show the effects of a combination of Compound A (i.e., 3-(2-cyanophenyl)-5-(2-pyridyl)-1-phenyl-1,2-dihydropyridin-2-one) and donepezil on dopamine turnover, DOPAC, HVA, DOPAC/DA and HVA/DA, respectively, in mice striatum.

FIG. 4 shows the effects of Compound A (i.e., 3-(2-cyanophenyl)-5-(2-pyridyl)-1-phenyl-1,2-dihydropyridin-2-one) and the combination of Compound A (i.e., 3-(2-cyanophenyl)-5-(2-pyridyl)-1-phenyl-1,2-dihydropyridin-2-one) and donepezil on kainate-induced neuronal toxicity in cortical neurons. The * indicates that p<0.05 vs. control, and # indicates that p<0.05 vs. donepezil alone.

FIG. 5 shows the effect of Compound A (i.e., 3-(2-cyanophenyl)-5-(2-pyridyl)-1-phenyl-1,2-dihydropyridin-2-one) and the combination treatment of Compound A (i.e., 3-(2-cyanophenyl)-5-(2-pyridyl)-1-phenyl-1,2-dihydropyridin-2-one) and donepezil on NMDA-induced neural toxicity in cortical neurons. The * indicates that p<0.05 vs. control.

### DETAILED DESCRIPTION OF THE INVENTION

“Patient” refers to animals, preferably mammals, more preferably humans. The term “patient” includes men and women; and includes adults, children and neonates. In one embodiment, the patient can be an animal companion, such as a dog or a cat.

“Cognitive impairments” refer to an acquired deficit in one or more of memory function, problem solving, orientation, and abstraction that impinges on a patient's ability to function independently.

“Dementia” refers to a global deterioration of intellectual functioning in clear consciousness, and is characterized by one or more symptoms of disorientation, impaired memory, impaired judgment, and impaired intellect.

“Administered separately” with reference to the administration of two or more compounds to treat and/or prevent the diseases and disorders described herein includes, for example, the sequential administration of the compounds in any order or the simultaneous administration of the compounds. Simultaneous administration of the compounds means that the compounds are administered to the patient at substantially the same time or at exactly the same time, depending on the mode of administration. The sequential administration of the compounds may occur in any order and may occur with any amount of time elapsing between administration of the compounds. Sequential administration may be based on factors that would influence which of the compounds should be administered first and which should be administered second, and how much time should elapse between administration of the compounds. For example, when two or more compounds are administered separately and sequentially, factors that effect when the compounds are administered to the patient include, for example, (a) the time(s) that provides the best efficacy for the compound being administered, (b) the time(s) that provides the fewest side effects for the compound being administered, (c) the dosage of the compound, (d) the route of administration of the compound, (e) the disease or disorder being treated, (f) the patient being treated, (g) the in vivo relationship of the compounds being administered, and other such factors known in the art. Preferably, the time intervals for sequential administration are chosen so that the effect on the disease or disorder being treated in the combined use of the active ingredients is greater than additive when compared to the effect which would be obtained by use of only one of the active ingredients.

The term “combination” refers to the AMPA receptor antagonist (e.g., 1,2-dihydropyridine compound) and the second active ingredient (e.g., nootropic, such as cholinesterase inhibitors) being administered separately as distinct pharmaceutical compositions or formulations (e.g., a first pharmaceutical composition comprising an AMPA receptor antagonist and a second pharmaceutical composition comprising a nootropic). The pharmaceutical compositions or formulations can have the same or different modes of administration.

“Active ingredient” refers to the 1,2-dihydropyridines and cholinesterase inhibitors described herein that are responsible for treatment and/or prophylaxis of a disease or disorder.

“Monotherapy” is a therapy which uses only one active ingredient for treatment and/or prophylaxis of a disease or disorder.

“Combination therapy” is a therapy where two or more active ingredients are administered separately or are administered in the form of a pharmaceutical composition for the treatment and/or prophylaxis of a disease.

“Therapeutically effective amount” refers to the amount of the active ingredient that is necessary for the treatment and/or prophylaxis of a disease. When two or more active ingredients are administered for combination therapy, the term “therapeutically effective amount” refers to the amount of active ingredients that are necessary for treatment and/or prophylaxis of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in combination therapy to achieve a result that would be equal to or greater than the result that the second active ingredient would achieve if it was used for monotherapy); (b) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in combination therapy to achieve a result that would be equal to or greater than the result that the first active ingredient would achieve if it was used for monotherapy; and the sub-therapeutic amount of the second active ingredient can be used in combination therapy to achieve a result that would be equal to or greater than the result that the second active ingredient would achieve if it was used for monotherapy).

“Commercial packages,” also known as kits, can include a combination of (i) a first pharmaceutical composition or formulation comprising the AMPA receptor antagonist (e.g., 1,2-dihydropyridine compound); (ii) a second pharmaceutical composition or formulation comprising the second active ingredient (e.g., nootropic, such as cholinesterase inhibitors); (iii) instructions approved by the FDA for using the pharmaceutical compositions or formulations for treating or preventing the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The commercial package can supply enough medication and materials for days, weeks or months. In another embodiment, “commercial packages” can include (i) pharmaceutical composition or formulation comprising both the AMPA receptor antagonist (e.g., 1,2-dihydropyridine compound) and the second active ingredient (e.g., nootropic, such as cholinesterase inhibitors); (ii) instructions approved by the FDA for using the pharmaceutical composition or formulation for treating or preventing the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The commercial package can supply enough medication and materials for days, weeks or months.

“Hydrate” refers to a compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as compound.¼H₂O; compound.½H₂O; compound.¾H₂O; compound.2H₂O; compound.5½ H₂O; compound.6H₂O; and the like. The “compound” can be any described herein, such as 3-(2-cyanophenyl)-5-(2-pyridyl)-1-phenyl-1,2-dihydropyridin-2-one.

“Pharmaceutically acceptable salts” are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the compounds of the invention can be made in the form of any other pharmaceutically acceptable salt.

In one embodiment, the compounds used in the methods and compositions described herein are AMPA receptor antagonists. The AMPA receptor antagonist may be any known in the art. Exemplary AMPA receptor antagonists include 1,2-dihydropyridine compounds, and quinoxaline-dione aminoalkylphosphonates. Preferably, the AMPA receptor antagonist is a 1,2-dihydropyridine compound. The 1,2-dihydropyridine compound used in the methods and compositions described herein may be any known in the art. The term “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

Preferably, the 1,2-dihydropyridine compound used in the methods and compositions described herein is a compound of Formula (I):

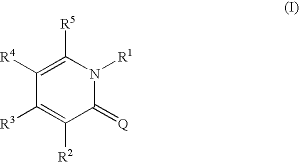

wherein

Q is NH, O or S;

R¹, R², R³, R⁴ and R⁵ are each independently hydrogen, halogen, C₁₋₆ alkyl, or —X-A;

X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²>, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring; provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

In one embodiment, the following compounds are excluded from the scope of the compound of Formula (I): (1) when Q is O; R¹ and R⁵ are hydrogen; and R², R³ and R⁴ are phenyl; (2) when Q is O; R¹ and R⁴ are hydrogen; and R², R³ and R⁵ are phenyl; and (3) when Q is O; R¹ and R² are hydrogen; and R³, R⁴ and R⁵ are phenyl.

In another embodiment preferred embodiment, the 1,2-dihydropyridine compound used in the methods and compositions described herein is a compound of Formula (II):

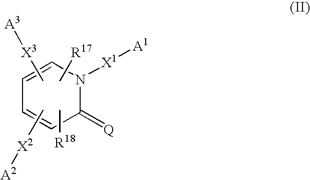

wherein

Q is NH, O or S;

X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and

R¹⁷ and R¹⁸ are each independently hydrogen, halogen, or C₁₋₆ alkyl.

In another embodiment, the invention provides the compound of Formula (II) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰), —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O, —O—CH₂—, —N(R¹³)—CO—N(R¹⁴)— and —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ allyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino, N—C₂₋₆ alkenyl-N—C₂₋₆alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkenylcarbonyloxy, N—C₁₋₆ alkylcarbamoyl, N—C₂₋₆ alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, N—C₁₋₆alkyl-N—C₂₋₆ alkenylamino, N—C₁₋₆ alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆ alkylsulfinyl; C₂₋₆ alkenylsulfinyl; C₂₋₆ alkynylsulfinyl; formyl; optionally substituted C₃₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (II) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (II) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (II) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (II) wherein A¹, A² and A³ are each independently selected from:

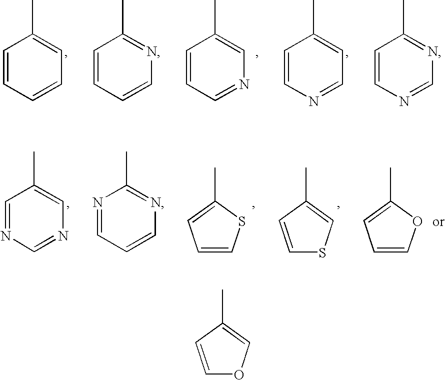

each of which may optionally be substituted. In another embodiment, the invention provides the compound of Formula (II) wherein A¹, A² and A³ are each independently substituted with hydroxyl, halogen, amino, or nitrile. In another embodiment, the invention provides the compound of Formula (II) wherein A¹, A² and A³ are each independently hydroxyl, halogen, amino, nitrile, or nitro. In another embodiment, the invention provides the compound of Formula (II) wherein Q is oxygen.

In another embodiment, the invention provides the compounds of Formula (I) or (II) wherein X¹, X² and X³ are each independently a single bond, —CH₂—, —CH(OH)—, —CH₂—CH₂—, —CH═CH—, —C≡C—, —O— or —CO—. In another embodiment, the invention provides the compounds of Formula (I) or (II) wherein X¹, X² and X³ are each a single bond. In another embodiment, the invention provides the compounds of Formula (I) or (II) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (I) or (II) wherein R¹⁷ and R¹⁸ are each hydrogen.

With respect to the 1,2-dihydropyridine compounds of the invention, the halogen atom indicates fluorine, chlorine, bromine, iodine and the like, and the preferable atoms include fluorine, chlorine and bromine.

With respect to the 1,2-dihydropyridine compounds of the invention, the C₁₋₆ alkyl indicates an alkyl having 1 to 6 carbons, and examples include linear chain or branched chain alkyl groups such as methyl, ethyl, n-propyl, iso-propyl, n-butyl, iso-butyl, sec-butyl (1-methylpropyl), tert-butyl, iso-pentyl, n-pentyl, tert-pentyl (1,1-dimethylpropyl), 1,2-dimethylpropyl, 2,2-dimethylpropyl(neopentyl), 1-ethylpropyl, 2 methylbutyl, n-hexyl, iso-hexyl, 1,2-dimethylbutyl, 1-ethyl-1-methylpropyl, 1-ethyl-2-methylpropyl, 1,1,2-trimethylpropyl, 1,2,2-trimethylpropyl, 1-ethylbutyl, 1-methylbutyl, 1,1-dimethylbutyl, 2,2-dimethylbutyl, 1,3-dimethylbutyl, 2,3-dimethylbutyl, 3,3-dimethylbutyl, 2-ethylbutyl, 1-methylpentyl, 2-methylpentyl, 3-methylpentyl, and the like.

With respect to the 1,2-dihydropyridine compounds of the invention, the C₂₋₆ alkenyl indicates an alkenyl group having 2 to 6 carbons, and examples include vinyl, 1-ethylethenyl (1-buten-2-yl), allyl (2-propenyl), 1-propenyl, iso-propenyl, 2-methyl-1-propenyl, 1-methyl-1-propenyl (2-buten-2-yl), 2-methyl-2-propenyl, 1-methyl-2-propenyl, 1-butenyl (1-buten-1-yl), 2-butenyl (2-buten-1-yl), 3-butenyl, 1-pentenyl, 1-hexenyl, 1,3-hexadienyl, 1,6-hexadienyl, and the like.

With respect to the 1,2-dihydropyridine compounds of the invention, the C₂₋₆ alkynyl indicates an alkynyl group having 2 to 6 carbons, and examples include ethynyl, 1-propynyl, 2-propynyl, 1-butynyl, 2-butynyl, 3-butynyl, 1-ethyl-1-propynyl, 1-ethynyl-2-propynyl, 2-methyl-3-butenyl, 1-pentynyl, 1-hexynyl, 1,3-hexadiynyl, 1,6-hexadiynyl, and the like.

With respect to the 1,2-dihydropyridine compounds of the invention, the C₁₋₆ alkoxy indicates an alkoxy group having 1 to 6 carbons, and examples include methoxy, ethoxy, n-propoxy, iso-propoxy, n-butoxy, iso-butoxy, sec-butoxy, tert-butoxy, n-pentyloxy, iso-pentyloxy, tert-pentyloxy, 1,2-dimethylpropoxy, neopentyloxy, 1-ethylpropoxy, 1-methylbutoxy, 2-methylbutyoxy, n-hexyloxy, iso-hexyloxy, 1-ethyl-1-methylpropoxy, 1-ethyl-2-methylpropoxy, 1,1,2-trimethylpropoxy, 1,2,2-trimethylpropoxy, 1,1-dimethylbutoxy, 2,2-dimethylbutoxy, 2,3-dimethylbutoxy, 3,3-dimethylbutoxy, 2-ethylbutoxy, 1,3-dimethylbutoxy, 1-ethylbutoxy, 1-methylbutoxy, 1-methylpentyloxy, 2-methylpentyloxy, 3-methylpentyloxy, and the like.

With respect to the 1,2-dihydropyridine compounds of the invention, the C₂₋₆ alkynyloxy indicates an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

With respect to the 1,2-dihydropyridine compounds of the invention, the C₂₋₆ alkenyloxy indicates an alkenyloxy group having 2 to 6 carbons, and examples include vinyloxy, 1-ethylethenyloxy(1-buten-2-yloxy), allyloxy(2-propenyloxy), 1-propenyloxy, iso-propenyloxy, 2-methyl-1-propenyloxy, 1-methyl-1-propenyloxy(2-buten-2-yloxy), 2-methyl-2-propenyloxy, 1-methyl-2-propenyloxy(1-buten-3-yloxy), 1-butenyloxy(1-buten-1-yloxy), 2-butenyloxy(2-buten-1-yloxy), 3-butenyloxy, 1-pentenyloxy, 1-hexenyloxy, 1,3-hexadienyloxy, 1,6-hexadienyloxy, and the like.

With respect to the 1,2-dihydropyridine compounds of the invention, the C₃₋₈ cycloalkyl indicates a cycloalkyl group composed of 3 to 8 carbon atoms, and examples include cyclopropyl, cyclobutyl, cyclopentyl, cyclohexyl, cycloheptyl, cyclooctyl, and the like.

With respect to the 1,2-dihydropyridine compounds of the invention, the C₃₋₈ cycloalkenyl indicates a cycloalkenyl group composed of 3 to 8 carbon atoms, and examples include cyclopropen-1-yl, 2-cyclopropen-1-yl, cyclobuten-1-yl, 2-cyclobuten-1-yl, 1,3-cyclobutadien-1-yl, cyclopenten-1-yl, 2-cyclopenten-1-yl, 3-cyclopenten-1-yl, 1,3-cyclopentadien-1-yl, 1,4-cyclopentadien-1-yl, 2,4-cyclopentadien-1-yl, cyclohexen-1-yl, 2-cyclohexen-1-yl, 3-cyclohexen-1-yl, 1,3-cyclohexadien-1-yl, 1,4-cyclohexadien-1-yl, 1,5-cyclohexadien-1-yl, 2,4-cyclohexadien-1-yl, 2,5-cyclohexadien-1-yl, cyclohepten-1-yl, 2-cyclohepten-1-yl, 3-cyclohepten-1-yl, 4-cyclohepten-1-yl, 1,3-cyclopentadien-1-yl, 1,4-cyclopentadien-1-yl, 1,5-cycloheptadien-1-yl, 1,6-cycloheptadien-1-yl, 2,4-cycloheptadien-1-yl, 2,5-cycloheptadien-1-yl, 2,6-cycloheptadien-1-yl, 3,5-cycloheptadien-1-yl, 1,3,5-cycloheptatrien-1-yl, 1,3,6-cycloheptatrien-1-yl, 1,4,6-cycloheptatrien-1-yl, 2,4,6-cycloheptatrien-1-yl, cycloocten-1-yl, 2-cycloocten-1-yl, 3-cycloocten-1-yl, 4-cycloocten-1-yl, 1,3-cyclooctadien-1-yl, 1,4-cyclooctadien-1-yl, 1,5-cyclooctadien-1-yl, 1,6-cyclooctadien-1-yl, 1,7-cyclooctadien-1-yl, 2,4-cyclooctadien-1-yl, 2,5-cyclooctadien-1-yl, 2,6-cyclooctadien-1-yl, 2,7-cyclooctadien-1-yl, 3,5-cyclooctadien-1-yl, 3,6-cyclooctadien-1-yl, 1,3,5-cyclooctatrien-1-yl, 1,3,6-cyclooctatrien-1-yl, 1,3,7-cyclooctatrien-1-yl, 1,4,6-cyclooctatrien-1-yl, 1,4,7-cyclooctatrien-1-yl, 1,5,7-cyclooctatrien-1-yl, 2,4,6-cyclooctatrien-1-yl, 2,4,7-cyclooctatrien-1-yl group, and the like.

With respect to the 1,2-dihydropyridine compounds of the invention, the 5- to 14-membered non-aromatic heterocyclic ring means a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolinyl, piperidyl, piperazinyl, pyrazolidinyl, imidazolidinyl, morpholinyl, tetrahydrofuryl, tetrahydropyranyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

With respect to the 1,2-dihydropyridine compounds of the invention, the C₆₋₁₄ aromatic hydrocarbocyclic ring and the aryl mean an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

With respect to the 1,2-dihydropyridine compounds of the invention, the 5- to 14-membered aromatic heterocyclic ring and the heteroaryl ring mean mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinolizinyl, phthalazyl, naphthylidinyl, quinoxalyl, quinazolinyl, cinnolinyl, pteridinyl, imidazotriazinyl, pyrazinopyridazinyl, acridinyl, phenanthridinyl, carbazolyl, carbazolinyl, perimidinyl, phenanthrolinyl, phenazinyl, imidazopyridyl, imidazopyrimidinyl, or pyrazolopyridyl; (2) aromatic heterocyclic rings containing sulfur such as thienyl or benzothienyl; (3) aromatic heterocyclic rings containing oxygen such as furyl, pyranyl, cyclopentapyranyl, benzofuryl, or iso-benzofuryl; and (4) aromatic heterocyclic rings containing 2 or more different hetero atoms such as thiazolyl, iso-thiazolyl, benzothiazolyl, benzothiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

In another embodiment, the 1,2-dihydropyridine compound used in the methods and compositions described herein is preferably a compound of Formula (III):

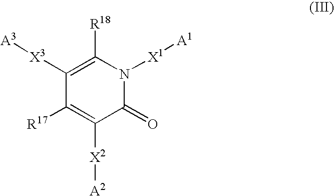

wherein X¹, X², X³, A¹, A², A³, R¹⁷ and R¹⁸ have the same meanings as defined in the above compound of Formula (II).

In another embodiment, the invention provides the compounds of Formula (III) wherein A¹, A² and A³ are each independently an optionally substituted C₆₋₁₄ aromatic hydrocarbon ring or 5- to 14-membered aromatic hetero ring. In another embodiment, the invention provides the compounds of Formula (III) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (III) wherein A¹, A² and A³ are each independently selected from:

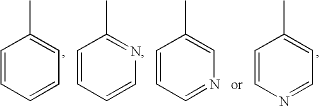

each of which may optionally be substituted. In another embodiment, the invention provides the compounds of Formula (III) wherein the bonding site of the substituent at A¹, A² and A³ are in the α-position of the carbon atom bonding to the group X¹, X² and X³, respectively. In another embodiment, the invention provides the compounds of Formula (III) wherein X¹, X² and X³ are single bonds. In another embodiment, the invention provides the compounds of Formula (III) wherein R⁷ and R¹⁸ are hydrogen.

In one embodiment, the 1,2-dihydropyridine compound used in the methods and compositions described herein is preferably Compound A:

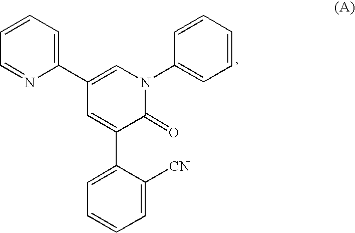

The IUPAC name for Compound A is 2-(2-oxo-1-phenyl-5-pyridin-2-yl-1,2-dihydropyridin-3-yl)benzonitrile. Compound A may also be referred to as 3-(2-cyanophenyl)-5-(2-pyridyl)-1-phenyl-1,2-dihydropyridin-2-one.

Throughout the specification, the terms “Compound A,” “2-(2-oxo-1-phenyl-5-pyridin-2-yl-1,2-dihydropyridin-3-yl)benzonitrile,” and “3-(2-cyanophenyl)-5-(2-pyridyl)-1-phenyl-1,2-dihydropyridin-2-one” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates thereof, and stereoisomer of hydrates of pharmaceutically acceptable salts thereof. In another embodiment, the terms “Compound A,” “2-(2-oxo-1-phenyl-5-pyridin-2-yl-1,2-dihydropyridin-3-yl)benzonitrile,” and “3-(2-cyanophenyl)-5-(2-pyridyl)-1-phenyl-1,2-dihydropyridin-2-one” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2 pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3-dimethylaminophenyl)-1,2-dihydropyridin-2-one; 3-(2-cyanophenyl)-5-(2-pyridyl)-1-[3-(5-methoxymethyl-2-oxazolidinone-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1-(4-methylthiophenyl)-1,2-dihydropyridin-2-one; 3-(2-cyanophenyl)-5-(2-pyridyl)-1-(4-methylsulfonylphenyl-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-1-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one; 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one; 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyridin-2-one; 3-(2-cyanophenyl)-5-(2-pyridyl)-1-(pyrimidin-5-yl)-1,2-dihydropyridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 3-(2-chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluene-4-yl)-1-,2-dihydropyridin-2-one; 3-(2-cyanophenyl)-1-[3-(dimethylaminoethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidinoethoxy)phenyl]-5-(2-pyridyl)-1,2-dihydropyridin-2-one; 3-(2 cyanophenyl)-1-[3-(diisopropylaminoethoxy)phenyl]-5-(-2-pyridyl)-1,2-dihydropyridin-2-one; 3-(2-cyanophenyl)-1-[3-(4-piperidinobutyl-1-oxy)phenyl]-5-(2-pyridyl)-1,2-dihydropyridin-2-one; 3-(2-cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3-(2-cyanophenyl)-1-(3-pyridyl)-5-(2-pyrimidinyl) 1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyridin-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridine-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morpholinopyridin-6-yl)-1-phenyl-1,2-dihydropyridin-2-one; 3-(2-cyanophenyl)-1-(3-hydroxyphenyl)-5-(2-pyridyl)-1,2-dihydropyridin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesulfonyl)piperidin-4-yl-oxy]phenyl}-5-(2-pyridyl)-1,2-dihydropyridin-2-one; 1-[3-(N-methylpiperidin-4-yl-oxy)phenyl]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluene-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazolyl)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication No. WO 03/047577, WO 04/009553, WO 06/004100, and WO 06/004107, the disclosures of which are incorporated by reference herein in their-entirety. Methods for making other AMPA receptor antagonists are described in WO 2005/094797.

In one embodiment, the compounds used in the methods and compositions described herein are nootropics. Nootropic compounds include cholinesterase inhibitors. The cholinesterase inhibitors used in the methods and compositions of the invention may be any known in the art. The term “cholinesterase inhibitor” includes cholinesterase inhibitors, pharmaceutically acceptable salts of cholinesterase inhibitors, stereoisomers of cholinesterase inhibitors, and pharmaceutically acceptable salts of stereoisomers of cholinesterase inhibitors. Exemplary cholinesterase inhibitors include donepezil, tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, eptastigmine, upreazine, 3-[1-(phenylmethyl)-4-piperidinyl]-1-(2,3,4,5-tetrahydro-1H-1-benzazepin-8-yl)-1-propanone, (2-[2-(1-benzylpiperidin-4-yl)ethyl]-2,3-dihydro-9-methoxy-1H-pyrrolo[3,4-b]quinolin-1-one), and the like. In one embodiment, the cholinesterase inhibitor is donepezil, tacrine, galantamine, or rivastigmine.

In one embodiment, the preferred cholinesterase inhibitors used in the methods and compositions described herein are compounds of formula (IV):

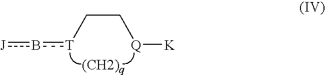

wherein J is (a) a substituted or unsubstituted group selected from (i) phenyl, (ii) pyridyl, (iii) pyrazyl, (iv) quinolyl, (v) cyclohexyl, (vi) quinoxalyl, and (vii) furyl; (b) a monovalent or divalent group, in which the phenyl can have one or more substituents selected from (i) indanyl, (ii) indanonyl, (iii) indenyl, (iv) indenonyl, (v) indanedionyl, (vi) tetralonyl, (vii) benzosuberonyl, (viii) indanolyl, and (ix) C₆H₅—CO—CH(CH₃)—; (c) a monovalent group derived from a cyclic amide compound; (d) a lower alkyl; or (e) R²¹—CH═CH—, in which R²¹ is hydrogen or a lower alkoxycarbonyl; B is —(CHR²²)ᵣ—, —CO—(CHR²²)ᵣ—, NR⁴—(CHR²²)ᵣ—, —CO—NR⁵—(CHR²²)—, —CH═CH—(CHR²²), —OCOO—(CHR²²)ᵣ—, —OOC—NH—(CHR²²)ᵣ—, NH—CO—(CHR²²)ᵣ, —CH₂—CO—NH—(CHR²²)—, (CH₂)₂—NH—(CHR²²)ᵣ, —CH(OH)—(CHR²²)—, ═(CH—CH═CH)_{b}—, ═CH—(CH₂)ᵣ—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbonyl; R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of 1 to 10; R²² is hydrogen or methyl so that one alkylene group can have no methyl branch or one or more methyl branches; b is an integer of 1 to 3; c is zero or an integer of 1 to 9; d is zero or an integer of 1 to 5; T is nitrogen or carbon; Q is nitrogen, carbon or

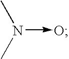

q is an integer of 1 to 3; K is hydrogen, phenyl, substituted phenyl, arylalkyl in which the phenyl can have a substituent, cinnamyl, a lower alkyl, pyridylmethyl, cycloalkylalkyl, adamantanemethyl, furylmethyl, cycloalkyl, lower alkoxycarbonyl or an acyl; and [symbol] is a single bond or a double bond.

In the compound of formula (IV), J is preferably (a) or (b), more preferably (b). In the definition of (b), a monovalent group (2), (3) and (5) and a divalent group (2) are preferred. The group (b) preferably includes the formulae shown below:

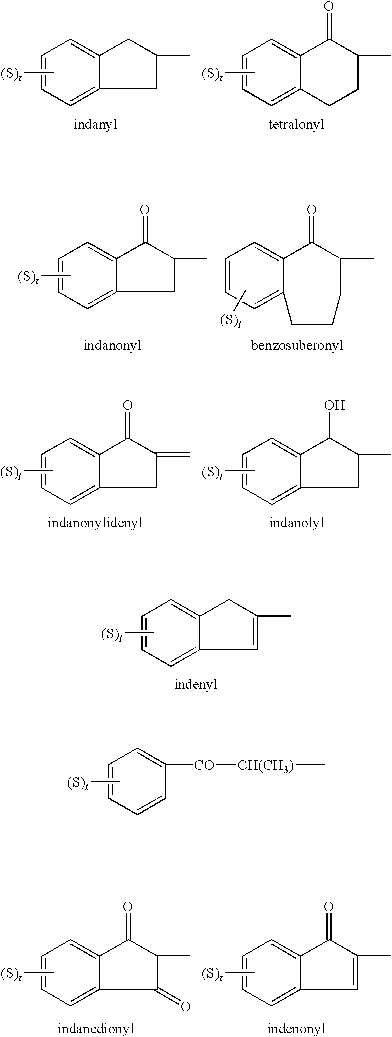

wherein t is an integer of 1 to 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy thereon. (S)ₜ can form methylene dioxy or ethylene dioxy on two-adjacent carbon atoms of the phenyl. Of the above groups, indanonyl, indanedionyl and indenyl, optionally having substituents on the phenyl, are the most preferred.

In the definition of B, —(CHR²²)ᵣ, —CO—(CHR²²)ᵣ—, ═(CH—CH═CH)_{b}—, ═CH—(CH₂)_{c}— and ═(CH—CH)_{d}═ (where b is an integer of 1 to 3; c is zero or an integer of 1 to 9; d is zero or an integer of 1 to 5) are preferable. The group —(CHR²²)ᵣ— in which R²² is hydrogen and r is an integer of 1 to 3, and the group ═CH—(CH₂)_{c}— are most preferable. The preferable groups of B can be connected with (b) of J, in particular (b)(2). The ring containing T and Q in formula (I) can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2. It is preferable that K is a phenyl, arylalkyl, cinnamyl, phenylalkyl or a phenylalkyl having a substituent(s) on the phenyl.

In one embodiment, the preferred cholinesterase inhibitors used in the methods and compositions described herein are compounds of formula (V):

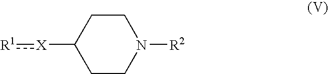

wherein R¹ is a (1) substituted or unsubstituted phenyl; (2) a substituted or unsubstituted pyridyl; (3) a substituted or unsubstituted pyrazyl; (4) a substituted or unsubstituted quinolyl; (5) a substituted or unsubstituted indanyl; (6) a substituted or unsubstituted cyclohexyl; (7) a substituted or unsubstituted quinoxalyl; (8) a substituted or unsubstituted furyl; (9) a monovalent or divalent group derived from an indanone having a substituted or unsubstituted phenyl; (10) a monovalent group derived from a cyclic amide compound; (11) a lower alkyl; or (12) R³—CH═C—, where R³ is a hydrogen atom or a lower alkoxycarbonyl; X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)—, —CH₂—C(O)—NH —(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl, a lower alkoxycarbonyl; n is an integer of 0 to 6; R⁴ is hydrogen, lower alkyl, acyl, lower alkylsulfonyl, a substituted or unsubstituted phenyl, or a substituted or unsubstituted benzyl; and R⁵ is hydrogen, lower alkyl, or phenyl; R² is a substituted or unsubstituted phenyl; a substituted or unsubstituted arylalkyl; a cinnamyl; lower alkyl; pyridylmethyl; cycloalkylalkyl; adamantanemethyl; or furylmethyl; and [symbol] is a single bond or a double bond.

With respect to the cholinesterase inhibitors described herein, the term “lower alkyl” means a straight or branched alkyl having 1 to 6 carbon atoms. Exemplary “lower alkyl” groups include methyl, ethyl, propyl, isopropyl, butyl, isobutyl, sec-butyl, tert-butyl, pentyl(amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimethyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl is preferably methyl, ethyl, propyl or isopropyl; more preferably methyl.

With respect to the cholinesterase inhibitors described herein, specific examples of the substituents for the substituted or unsubstituted phenyl, pyridyl, pyrazyl, quinolyl, indanyl, cyclohexyl, quinoxalyl and furyl in the definition of R¹ include lower alkyl groups having 1 to 6 carbon atoms, such as methyl, ethyl, n-propyl, isopropyl, n-butyl, isobutyl, and tert-butyl groups; lower alkoxy corresponding to the above-described lower alkyl, such as methoxy and ethoxy groups; nitro; halogen, such as chlorine, fluorine and bromine; carboxyl; lower alkoxycarbonyl corresponding to the above-described lower alkoxy, such as methoxycarbonyl, ethoxycarbonyl, isopropoxycarbonyl, n-propoxycarbonyl, and n-butyloxycarbonyl groups; amino; lower monoalkylamino; lower dialkylamino; carbamoyl; acylamino derived from aliphatic saturated monocarboxylic acids having 1 to 6 carbon atoms, such as acetylamino, propionylamino, butyrylamino, isobutyrylamino, valerylamino, and pivaloylamino; cycloalkyloxycarbonyl, such as a cyclohexyloxycarbonyl; lower alkylaminocarbonyl, such as methylaminocarbonyl and ethylaminocarbonyl; lower alkylcarbonyloxy corresponding to the above-defined lower alkyl, such as methylcarbonyloxy, ethylcarbonyloxy, and n-propylcarbonyloxy; halogenated lower alkyl, such as trifluoromethyl; hydroxyl; formyl; and lower alkoxy lower alkyl, such as ethoxymethyl, methoxymethyl and methoxyethyl. The “lower alkyl” and “lower alkoxyl” in the above description of the substituents include all the groups derived from the above-mentioned groups. There may be 1, 2 or 3 substituents, which may be the same or different.

With respect to the cholinesterase inhibitors described herein, when the substituent is phenyl, the following is within the scope of the substituted phenyl:

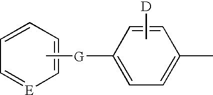

wherein G is —C(O)—, —O—C(O)—, —O—, —CH₂—NH—C(O)—, —CH₂—O—, —CH₂—SO₂—, —CH(OH)—, or —CH₂—S(→O)—; E is carbon or nitrogen; and D is a substituent.

With respect to the cholinesterase inhibitors described herein, preferred examples of the substituents (i.e., “D”) for the phenyl include lower alkyl, lower alkoxy, nitro, halogenated lower alkyl, lower alkoxycarbonyl, formyl, hydroxyl, and lower alkoxy lower alkyl, halogen, and benzoyl and benzylsulfonyl. There may be 2 or more substituents, which may be the same or different. Preferred examples of the substituent for the pyridyl include lower alkyl and amino and halogen. Preferred examples of the substituent for the pyrazyl include lower alkoxycarbonyl, carboxyl, acylamino, carbamoyl, and cycloalkyloxycarbonyl.

With respect to the cholinesterase inhibitors described herein, with respect to R¹, the pyridyl is preferably a 2-pyridyl, 3-pyridyl, or 4-pyridyl; the pyrazyl is preferably a 2-pyrazinyl; the quinolyl is preferably a 2-quinolyl or 3-quinolyl; the quinoxalinyl is preferably a 2-quinoxalinyl or 3-quinoxalinyl; and the furyl is preferably a 2-furyl.

With respect to the cholinesterase inhibitors described herein, examples of monovalent or divalent groups derived from an indanone having an unsubstituted or substituted phenyl of (A) or (B):

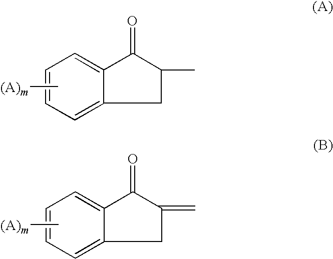

where m is an integer of from 1 to 4, and each A is independently hydrogen, lower alkyl, lower alkoxy, nitro, halogen, carboxyl, lower alkoxycarbonyl, amino, lower monoalkylamino, lower dialkylamino, carbamoyl, acylamino derived from aliphatic saturated monocarboxylic acids having 1 to 6 carbon atoms, cycloalkyloxycarbonyl, lower alkylaminocarbonyl, lower alkylcarbonyloxy, halogenated lower alkyl, hydroxyl, formyl, or lower alkoxy lower alkyl; preferably hydrogen, lower alkyl, or lower alkoxy; most preferably the indanone is unsubstituted or substituted with 1 to 3 methoxy.

With respect to the cholinesterase inhibitors described herein, examples of the monovalent group derived from a cyclic amide compound include quinazolone, tetrahydroisoquinolinone, tetrahydrobenzodiazepinone, and hexahydrobenzazocinone. The monovalent group can be any one having a cyclic amide in the structural formula thereof, and is not limited to the above-described specific examples. The cyclic amide can be one derived from a monocyclic or condensed heterocyclic ring. The condensed heterocyclic ring is preferably one formed by condensation with phenyl. In this case, phenyl can be substituted with a lower alkyl group having 1 to 6 carbon atoms, preferably methyl, or lower alkoxy having 1 to 6 carbon atoms, preferably methoxy.

With respect to the cholinesterase inhibitors described herein, examples of the monovalent group include the following:

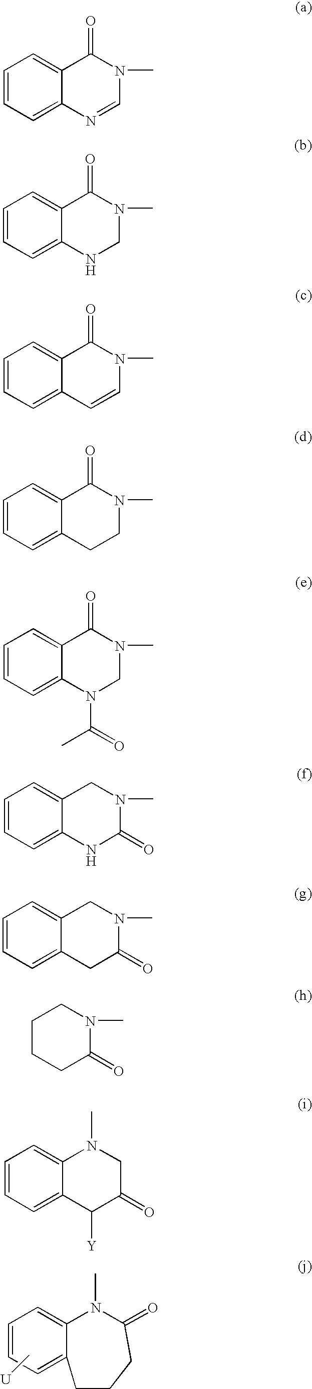

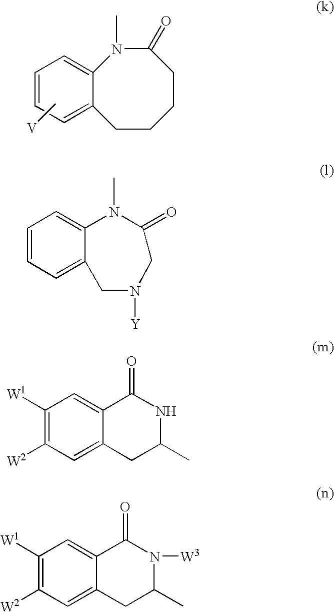

In the above formulae, Y is hydrogen or lower alkyl; V and U are each hydrogen or lower alkoxy (preferably dimethoxy); W¹ and W² are each hydrogen, lower alkyl, or lower alkoxy; and W³ is hydrogen or a lower alkyl. The right hand ring in formulae (j) and (1) is a 7-membered ring, while the right hand ring in formula (k) is an 8-membered ring.

With respect to the cholinesterase inhibitors described herein, the most preferred examples of R¹ include a monovalent group derived from an indanone having an unsubstituted or substituted phenyl and a monovalent group derived from a cyclic amide compound. With respect to the cholinesterase inhibitors described herein, the most preferred examples of X include —(CH₂)ₙ—, amide, or groups represented by the above formulae where n is 2. Thus, it is most preferred that any portion of a group represented by R¹ [symbol] X— have a carbonyl or amide.

With respect to the cholinesterase inhibitors described herein, the substituents involved in the expressions “a substituted or unsubstituted phenyl” and “a substituted or unsubstituted arylalkyl” in the above definition of R² are the same substituents as those described for the above definitions of phenyl, pyridyl, pyrazyl, quinolyl, indanyl, cyclohexyl, quinoxalyl or furyl in the definition of R¹. The term “arylalkyl” is intended to mean an unsubstituted benzyl or phenethyl or the like. Specific examples of the pyridylmethyl include 2-pyridylmethyl, 3-pyridylmethyl, and 4-pyridylmethyl. Preferred examples of R² include benzyl and phenethyl. The symbol [symbol] means a double or single bond. The bond is a double bond only when R¹ is the divalent group (B) derived from an indanone having an unsubstituted or substituted phenyl, while it is single bond in other cases.

In one embodiment, the preferred cholinesterase inhibitors used in the methods and compositions described herein are compounds of formula (VI):

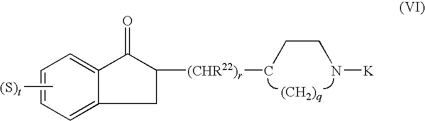

wherein r is an integer of 1 to 10; each R²² is independently hydrogen or methyl; K is a phenalkyl or a phenalkyl having a substituent on the phenyl; each S is independently hydrogen, C₁₋₆ lower alkyl, or C₁₋₆ lower alkoxy; t is an integer of 1 to 4; q is an integer of 1 to 3; with the proviso that (S)ₜ can be methylenedioxy or ethylenedioxy joined to two adjacent carbon atoms of the phenyl.

In other embodiments, the compound of formula (VI) is 1-benzyl-4-((5,6-dimethoxy-1-indanon)-2-yl)methylpiperidine; 1-benzyl-4-((5,6-dimethoxy-1-indanon)-2-ylidenyl)methyl-piperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy-1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; pharmaceutically acceptable salts of one or more of the foregoing; stereoisomers of one or more of the foregoing; or pharmaceutically acceptable salts of stereoisomers of one or more of the foregoing.

In other embodiments, the compound of formula (VI) used in the methods and compositions described herein is preferably 1-benzyl-4-((5,6-dimethoxy-1-indanon)-2-yl)methylpiperidine; which is represented by formula (B):

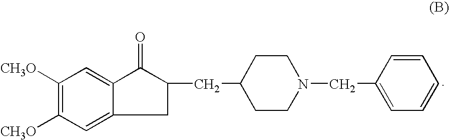

The compound of Formula (B), known as donepezil, may be in the form of a polymorph or a polymorphic crystal. For example, donepezil may be in the form of polymorph (II), (III), (IV), or (V); preferably polymorph (III). Donepezil may be in the form of polymorphic crystals (A), (B), or (C). Polymorphs, polymorphic crystals, and methods for making polymorphs and polymorphic crystals are described in U.S. Pat. Nos. 5,985,864, 6,140,321 and 6,245,911, the disclosures of which are incorporated by reference herein in their entirety.

In still other embodiments, the compound of formula (III) is 1-benzyl-4-((5,6-dimethoxy-1-indanon)-2-yl)methylpiperidine hydrochloride, which is also known as donepezil hydrochloride, and which is represented by formula (B1):

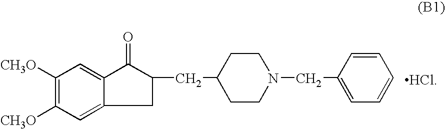

The compounds of the invention can have an asymmetric carbon atom(s), depending upon the substituents, and can have stereoisomers, which are within the scope of the invention. For example, donepezil or pharmaceutically acceptable salts thereof can be in the forms described in Japanese Patent Application Nos. 4-187674 and 4-21670, the disclosures of which are incorporated by reference herein in their entirety.

Japanese Patent Application No. 4-187674 describes a compound of formula (B2):

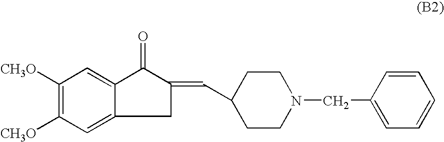

which can be in the form of a pharmaceutically acceptable salt, such as a hydrochloride salt. Japanese Patent Application No. 4-21670 describes compounds of formula (B3):

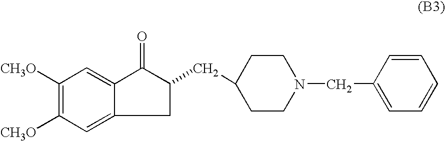

which can be in the form of a pharmaceutically acceptable salt, such as a hydrochloride salt; and compounds of formula (B4):

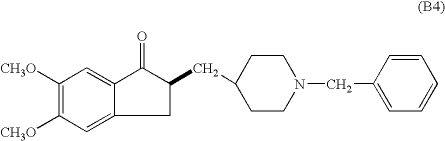

which can be in the form of a pharmaceutically acceptable salt, such as a hydrochloride salt; and compounds of formula (B5):

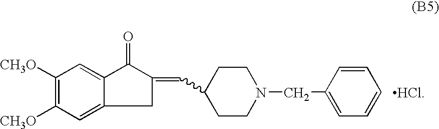

Throughout the specification, the terms “donepezil” and “1-benzyl-4-((5,6-dimethoxy-1-indanon)-2-yl)methylpiperidine” are intended to include one or more of the following (e.g., combinations of two or more thereof): pharmaceutically acceptable salts; stereoisomers; polymorphs; and polymorphic crystals.

The cholinesterase inhibitors of the invention are commercially available or can be prepared by processes known in the art, such as those described, for example, in U.S. Pat. Nos. 4,895,841, 5,985,864, 6,140,321 and 6,245,911; WO 98/39000, and Japanese Patent Application Nos. 4-187674 and 4-21670, the disclosures of which are incorporated by reference herein in their entirety.

The invention provides pharmaceutical compositions comprising a therapeutically effective amount of: (i) at least one AMPA receptor antagonist; (ii) at least one nootropic; and (iii) a pharmaceutically acceptable excipient. The AMPA receptor antagonist may be any known in the art, such as the 1,2-dihydropyridine compounds described herein. The nootropic may be any known in the art, such as the cholinesterase inhibitors described herein.

In other embodiments, the invention provides combinations comprising a therapeutically effective amount of: (i) at least one AMPA receptor antagonist and (ii) at least one nootropic. The combination may be administered separately (e.g., simultaneously, sequentially) to a patient to treat the diseases or disorders described herein (e.g., cognitive impairments, dementia and the like). The AMPA receptor antagonist may be any known in the art, such as the 1,2-dihydropyridine compounds described herein. The nootropic may be any known in the art, such as the cholinesterase inhibitors described herein.

In other embodiments, the invention provides commercial packages (e.g., kits) comprising a therapeutically effective amount of: (i) at least one AMPA receptor antagonist and (ii) at least one nootropic; and (ii) instructions for simultaneous, separate or sequential use thereof in the treatment of the diseases and disorders described herein. The AMPA receptor antagonist may be any known in the art, such as the 1,2-dihydropyridine compounds described herein. The nootropic may be any known in the art, such as the cholinesterase inhibitors described herein.

In other embodiments, the invention provides pharmaceutical compositions comprising a therapeutically effective amount of: (i) at least one 1,2-dihydropyridine compound, (ii) at least one cholinesterase inhibitor, and (iii) at least one pharmaceutically acceptable excipient. The invention also provides combinations comprising a therapeutically effective amount of: (i) at least one 1,2-dihydropyridine compound and (ii) at least one cholinesterase inhibitor; wherein the compounds may be administered separately (e.g., simultaneously, sequentially) to a patient to treat the diseases or disorders described. The invention provides commercial packages (e.g., kits) comprising a therapeutically effective amount of: (i) at least one 1,2-dihydropyridine compound, (ii) at least one cholinesterase inhibitor; and (iii) instructions for the simultaneous, separate or sequential use of (i) and (ii) in the treatment of the diseases and disorders described herein. The 1,2-dihydropyridine compound can be any described herein. For example, the 1,2-dihydropyridine compound can be a compound of Formula (I), a compound of Formula (II), a compound of Formula (III), or Compound A. The cholinesterase inhibitor can be any described herein. For example, the cholinesterase inhibitor can be a compound of Formula (IV), a compound of Formula (V), a compound of Formula (VI), a compound of Formula (B), a compound of Formula (B1), a compound of Formula (B2), a compound of Formula (B3), a compound of Formula (B4), or a compound of Formula (B5). In other embodiments, the cholinesterase inhibitor can be tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, eptastigmine, upreazine, 3-[1-(phenylmethyl)-4-piperidinyl]-1-(2,3,4,5-tetrahydro-1H-1-benzazepin-8-yl)-1-propanone, or (2-[2-(1-benzylpiperidin-4-yl)ethyl]-2,3-dihydro-9-methoxy-1H-pyrrolo[3,4-b]quinolin-1-one. In one embodiment, the invention provides pharmaceutical compositions comprising a therapeutically effective amount of: (i) donepezil; (ii) 3-(2-cyanophenyl)-5-(2-pyridyl)-1-phenyl-1,2-dihydropyridin-2-one; and (iii) at least one pharmaceutically acceptable excipient.

The invention provides (i) methods for treating one or more cognitive impairments in a patient in need thereof, (ii) methods for treating dementia in a patient in need thereof; and (iii) methods for delaying the onset of dementia or one more cognitive impairments in a patient in need thereof by administering a therapeutically effective amount of: (a) at least one 1,2-dihydropyridine compound and (b) at least one cholinesterase inhibitor. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil.

The methods for treating dementia include methods for treating the behavioral disturbances associated with dementia. Exemplary behavioral disturbances include sexual disinhibition, changes in activity, changes in interpersonal relationships, physical aggressiveness, physical non-aggressiveness (e.g., wandering), verbal aggressiveness, and verbal non-aggressiveness (e.g., repetitive vocalization).

The cause(s) of the cognitive impairment(s) or dementia may be known or unknown. For example, dementia and cognitive impairment(s) may be caused by Alzheimer's disease, Parkinson's disease, Huntington's disease, Pick's disease, Lewy body disease, vascular disease (e.g., cerebrovascular disease), HIV, AIDS, epilepsy, brain tumors, brain lesions, multiple sclerosis, Down's syndrome, Rett's syndrome, progressive supranuclear palsy, frontal lobe syndrome, schizophrenia, traumatic brain injuries (e.g., closed head injuries), post coronary artery by-pass graft surgery, electroconvulsive shock therapy, chemotherapy, radiation therapy, radiation exposure, encephalitis, meningitis, fetal alcohol syndrome, Korsakoff's syndrome, anoxic brain injury, cardiopulmonary resuscitation, diabetes, menopause, strokes, high cholesterol levels, or spinal cord disorders (e.g., spinal cord injury, spinal cord ischemia, spinal cord infarction, spinal cord convulsions). In one embodiment, the cognitive impairments may be mild cognitive impairments or age-associated cognitive impairments. For additional descriptions of cognitive impairments, dementia, and the causes of cognitive impairments and dementia, the disclosures of U.S. Pat. No. 6,458,807, US Publication No. 2006/0018839, and WO 2005/074535 are incorporated by reference herein in their entirety.

In another embodiment of the invention, the cognitive impairments may be canine cognitive impairments or dysfunctions (CCD), which are the age related deterioration of cognitive abilities in dogs characterized by behavioral changes that cannot be wholly attributed to general medical conditions. When preparing veterinary compositions, one skilled in the art would appreciate that the composition would contain a therapeutically effective amount of: (a) at least one 1,2-dihydropyridine compound (e.g., 3-(2-cyanophenyl)-5-(2-pyridyl)-1-phenyl-1,2-dihydropyridin-2-one), (b) at least one cholinesterase inhibitor (e.g., donepezil), and (c) a veterinarily acceptable carrier. The dosage amount for a dog can be easily achieved based on the dosages that are acceptable for humans and the size and weight of the dog.

In other embodiments, the invention provides (i) methods for treating a neurodegenerative disease; and (ii) methods for delaying the onset of a neurodegenerative disease in a patient in need thereof by administering a therapeutically effective amount of: (a) at least one 1,2-dihydropyridine compound and (b) at least one cholinesterase inhibitor. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil. The neurodegenerative disease may be any known in the art. Exemplary neurodegenerative diseases include Alzheimer's disease, Parkinson's disease, Huntington's disease, amyotrophic lateral sclerosis, Pick's disease, Lewy body disease, prion diseases (e.g., Creutzfeldt-Jakob disease), multiple sclerosis, epilepsy, strokes, and the like.

In other embodiments, the invention provides (i) methods for treating Alzheimer's disease; and (ii) methods for delaying the onset of Alzheimer's disease in a patient in need thereof by administering a therapeutically effective amount of: (a) at least on 1,2-dihydropyridine compound and (b) at least one cholinesterase inhibitor. The Alzheimer's disease may be mild, moderate, or severe. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil.

In other embodiments, the invention provides (i) methods for treating Parkinson's disease; and (ii) methods for delaying the onset of Parkinson's disease in a patient in need thereof by administering a therapeutically effective amount of: (a) at least on 1,2-dihydropyridine compound and (b) at least one cholinesterase inhibitor. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil.

In other embodiments, the invention provides (i) methods for treating Huntington's disease; and (ii) methods for delaying the onset of Huntington's disease in a patient in need thereof by administering a therapeutically effective amount of: (a) at least on 1,2-dihydropyridine compound and (b) at least one cholinesterase inhibitor. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil.

In other embodiments, the invention provides (i) methods for treating amyotrophic lateral sclerosis; and (ii) methods for delaying the onset of amyotrophic lateral sclerosis in a patient in need thereof by administering a therapeutically effective amount of: (a) at least on 1,2-dihydropyridine compound and (b) at least one cholinesterase inhibitor. The 1,2-dihydropyridine compound and the cholinesterase inhibitor can be administered separately or can be administered in the form of a composition. In one embodiment, the 1,2-dihydropyridine compound is 3-(2-cyanophenyl)-5-(2-pyridyl)-1-phenyl-1,2-dihydropyridin-2-one; and the cholinesterase inhibitor is donepezil.

The AMPA receptor antagonists, such as the 1,2-dihydropyridine compounds, and the nootropics, such as the cholinesterase inhibitors, can be administered orally, topically, parenterally, by inhalation (nasal or oral), or rectally in dosage unit formulations containing conventional nontoxic pharmaceutically acceptable carriers, adjuvants, and vehicles as desired. The term parenteral includes subcutaneous, intravenous, intramuscular, intrathecal, intrasternal injection, or infusion techniques.

The daily dose of the AMPA receptor antagonists, such as the 1,2-dihydropyridine compounds of the invention (e.g., 3-(2-cyanophenyl)-5-(2-pyridyl)-1-phenyl-1,2-dihydropyridin-2-one) is usually about 30 μg to 10 grams, preferably, 100 μg to 5 grams or, more preferably, 100 μg to 100 mg in the case of oral administration. For administration by injection, the daily dose is usually about 30 μg to 1 gram, preferably 100 μg to 500 mg or, more preferably, 100 μg to 30 mg. The compounds are administered once daily or in several portions a day. When used in the context of a dosage amount, the numerical weight refers to the weight of the AMPA receptor antagonist, such as the 1,2-dihydropyridine compounds of the invention, exclusive of any salt, counterion, hydrate, and the like. Therefore to obtain the equivalent of 500 milligrams of 3-(2-cyanophenyl)-5-(2-pyridyl)-1-phenyl-1,2-dihydropyridin-2-one, it would be necessary to use more than 500 milligrams of a pharmaceutically acceptable salt and/or hydrate of the compound, due to the additional weight of the pharmaceutically acceptable salt and/or hydrate.

The daily dose of the nootropic, such as the cholinesterase inhibitors of the invention (e.g., donepezil) is usually about 0.1 milligram to 100 milligrams, preferably 1 milligram to 50 milligrams, more preferably 5 milligrams to 25 milligrams. In other embodiment, the daily dose is from 10 milligrams to 20 milligrams; or from 5 milligrams to 10 milligrams. The compounds are administered once daily or in several portions a day. When used in the context of a dosage amount, the numerical weight refers to the weight of the nootropic, such as the cholinesterase inhibitors of the invention, exclusive of any salt, counterion, and the like. Therefore to obtain the equivalent of 10 milligrams of donepezil, it would be necessary to use more than 10 milligrams of donepezil hydrochloride, due to the additional weight of the hydrochloride.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxymethyl-cellulose, acacia, powdered tragacanth, sodium carboxymethylcellulose, polyoxy-ethylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

Solid dosage forms for oral administration can include chewing gum, capsules, tablets, sublingual tablets, powders, granules, and gels. In such solid dosage forms, the active compound can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings, preferably film coatings.

To make tablets, the compounds can be admixed with pharmaceutically acceptable carriers known in the art such as, for example, vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like.

Sublingual administration refers to the administration in the mouth (e.g., under the tongue, between the cheek and gum, between the tongue and roof of the mouth). The highly vascular mucosal lining in the mouth is a convenient location for the compounds to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the compounds described herein can be mixed with flavoring or sweetening agents. The packaging material can be plastic, coated paper, or any material that prevents water or moisture from reaching the granules and/or powder.

Liquid dosage forms for oral administration can include pharmaceutically acceptable emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the compounds can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

For administration by inhalation, the compounds can be delivered from an insufflator, a nebulizer or a pressured pack or other convenient mode of delivering an aerosol spray. Pressurized packs can include a suitable propellant. Alternatively, for administration by inhalation, the compounds can be administered in the form of a dry powder composition or in the form of a liquid spray.

Suppositories for rectal administration can be prepared by mixing the active compounds with suitable nonirritating excipients such as cocoa butter and polyethylene glycols that are solid at room temperature and liquid at body temperature. Alternatively, an enema can be prepared by for rectal administration of the compounds described herein.

For topical administration to the epidermis, the compounds can be formulated as ointments, creams or lotions, or as the active ingredient of a transdermal patch. The compounds can also be administered via iontophoresis or osmotic pump. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the compounds can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrable compositions can contain polyethylene glycol 400. To form ointments, the compounds can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The compounds can also be topically applied using a transdermal system, such as one of an acrylic-based polymer adhesive with a resinous crosslinking agent impregnated with the compounds described herein and laminated to an impermeable backing. For example, the compounds can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

The invention provides for the compounds to be administered nasally to a patient to treat the diseases and disorders described herein. “Administered nasally” or “nasal administration” is intended to mean that at least one compound is combined with a suitable delivery system for absorption across the nasal mucosa of a patient. Generally, lower doses of the compound can be used for nasal administration when compared, for example, to the dose required for the oral administration.

The compounds can be administered, for example, as nasal sprays, nasal drops, nasal suspensions, nasal gels, nasal ointments, nasal creams or nasal powders. The compounds can also be administered using nasal tampons or nasal sponges. The compounds can be brought into a viscous basis via systems conventionally used, for example, natural gums, methylcellulose and derivatives, acrylic polymers (carbopol) and vinyl polymers (polyvinylpyrrolidone). In the compositions, many other excipients known in the art can be added such as water, preservatives, surfactants, solvents, adhesives, antioxidants, buffers, bio-adhesives, viscosity enhancing agents and agents to adjust the pH and the osmolarity.

The nasal delivery systems can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In other embodiments, the nasal delivery system can be a powder formulation. Powder formulations include, for example, powder mixtures, powder microspheres, coated powder microspheres, liposomal dispersions and combinations thereof. Preferably, the powder formulation is powder microspheres. The powder microspheres are preferably formed from various polysaccharides and celluloses selected from starch, methylcellulose, xanthan gum, carboxymethylcellulose, hydroxypropyl cellulose, carbomer, alginate polyvinyl alcohol, acacia, chitosans, and mixtures of two or more thereof.

In certain embodiments, the particle size of the droplets of the aqueous and/or non-aqueous solution or of the powders delivered to the nasal mucosa can be, for example, about 0.1 micron to about 100 microns; from about 1 micron to about 70 microns; from about 5 microns to about 50 microns; or from about 10 microns to about 20 microns. The particle sizes can be obtained using suitable containers or metering devices known in the art. Exemplary devices include mechanical pumps in which delivery is made by movement of a piston; compressed air mechanisms in which delivery is made by hand pumping air into the container; compressed gas (e.g., nitrogen) techniques in which delivery is made by the controlled release of a compressed gas in the sealed container; liquefied propellant techniques in which a low boiling liquid hydrocarbon (e.g., butane) is vaporized to exert a pressure and force the composition through the metered valve; and the like. Powders may be administered, for example, in such a manner that they are placed in a capsule that is then set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In one embodiment, the invention provides a nasally administrable pharmaceutical composition comprising at least one compound dispersed in a nasal delivery system that improves the solubility of the compound. The nasal delivery system that improves solubility can include one of the following or combinations thereof: (i) a glycol derivative (e.g., propylene glycol, polyethylene glycol, mixtures thereof); (ii) a sugar alcohol (e.g., mannitol, xylitol, mixtures thereof); (iii) glycerin; (iv) a glycol derivative (e.g., propylene glycol, polyethylene glycol or mixtures thereof) and glycerin; (v) ascorbic acid and water; (vi) sodium ascorbate and water; or (vii) sodium metabisulfite and water.

In another embodiment, the invention provides a nasally administrable pharmaceutical composition comprising at least one compound described herein and a nasal delivery system, where the nasal delivery system comprises at least one buffer to maintain the pH of the compound described herein, at least one pharmaceutically acceptable thickening agent and at least one humectant. The nasal delivery system can optionally further comprise surfactants, preservatives, antioxidants, bio-adhesives, pH adjusting agents, isotonicity agents, solubilizing agents, and/or other pharmaceutically acceptable excipients. The compound described herein can optionally be dispersed in a nasal delivery system that improves its solubility.

In another embodiment, the invention provides a nasally administrable pharmaceutical composition comprising at least one compound described herein and a nasal delivery system, where the nasal delivery system comprises at least one solubilizing agent, at least one pharmaceutically acceptable thickening agent and at least one humectant. The nasal delivery system can optionally further comprise buffers, pH adjusting agents, isotonicity agents, surfactants, preservatives, antioxidants, bio-adhesives, and/or other pharmaceutically acceptable excipients. The compound described herein can optionally be dispersed in a nasal delivery system that improves its solubility.

In another embodiment, the invention provides a nasally administrable pharmaceutical composition comprising at least one compound described herein and a nasal delivery system, where the nasal delivery system comprises at least one buffer to maintain the pH of the compound, at least one pharmaceutically acceptable thickening agent, at least one humectant, and at least one surfactant. The nasal delivery system can optionally further comprise pH adjusting agents, isotonicity agents, solubilizing agents, preservatives, antioxidants, bio-adhesives, and/or other pharmaceutically acceptable excipients. The compound described herein can optionally be dispersed in a nasal delivery system that improves its solubility.

In yet another embodiment, the invention provides a nasally administrable pharmaceutical composition comprising at least one compound described herein and a nasal delivery system, where the nasal delivery system comprises at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The compound can optionally be dispersed in a nasal delivery system that improves its solubility.

In yet another embodiment, the invention provides a nasally administrable pharmaceutical composition comprising at least one compound described herein and a nasal delivery system, where the nasal delivery system comprises at least one buffer to maintain the pH of the compound, at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The compound described herein can optionally be dispersed in a nasal delivery system that improves its solubility.

The buffer has a pH that is selected to optimize the absorption of the 1,2-dihydropyridine compound across the nasal mucosa. The particular pH of the buffer can vary depending upon the particular nasal delivery formulation as well as the specific compound selected. Buffers that are suitable for use in the invention include acetate (e.g., sodium acetate), citrate (e.g., sodium citrate dihydrate), phthalate, borate, prolamine, prolamine, carbonate, phosphate (e.g., monopotassium phosphate, disodium phosphate), and mixtures of two or more thereof.

The pH of the compositions should be maintained from about 3.0 to about 10.0. Compositions having a pH of less than about 3.0 or greater than about 10.0 can increase the risk of irritating the nasal mucosa of the patient. Further, it is preferable that the pH of the compositions be maintained from about 3.0 to about 9.0. With respect to the non-aqueous nasal formulations, suitable forms of buffering agents can be selected such that when the formulation is delivered into the nasal cavity of a mammal, selected pH ranges are achieved therein upon contact with, e.g., a nasal mucosa.

The solubilizing agent for use in the compositions of the invention can be any known in the art, such as carboxylic acids and salts thereof. Exemplary carboxylic acid salts include acetate, gluconate, ascorbate, citrate, fumarate, lactate, tartrate, maleate, maleate, succinate, or mixtures of two or more thereof.

The viscosity of the compositions of the present invention can be maintained at a desired level using a pharmaceutically acceptable thickening agent. For example, the viscosity may be at least 1000 cps; from about 1000 to about 10,000 cps; from about 2000 cps to about 6500 cps; or from about 2500 cps to about 5000 cps. Thickening agents that can be used in accordance with the present invention include, for example, methyl cellulose, xanthan gum, carboxymethyl cellulose, hydroxypropyl cellulose, carbomer, polyvinyl alcohol, alginates, acacia, chitosans, and mixtures of two or more thereof. The concentration of the thickening agent will depend upon the agent selected and the viscosity desired. Such agents can also be used in a powder formulation.

The nasally administrable compositions can also include a humectant to reduce or prevent drying of the mucus membrane and to prevent irritation thereof. Suitable humectants that can be used include, for example, sorbitol, mineral oil, vegetable oil and glycerol; soothing agents; membrane conditioners; sweeteners; and mixtures of two or more thereof. The concentration of the humectant will vary depending upon the agent selected. In one embodiment, the humectant can be present in the nasal delivery system in a concentration ranging from about 0.01% to about 20% by weight of the composition.

In other embodiments, the nasal delivery system can further comprise surfactants which enhance the absorption of the compound. Suitable surfactants include non-ionic, anionic and cationic surfactants. Exemplary surfactants include oleic acid, polyoxyethylene derivatives of fatty acids, partial esters of sorbitol anhydride, such as for example, Tweens (e.g., Tween 80, Tween 40, Tween 20), Spans (e.g., Span 40, Span 80, Span 20), polyoxyl 40 stearate, polyoxy ethylene 50 stearate, fasieates, bile salts, octoxynol, and mixtures of two or more thereof. Exemplary anionic surfactants include salts of long chain hydrocarbons (e.g., C₆₋₃₀ or C 10-20) having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetradecyl sulfate. One particularly preferred anionic surfactant is sodium lauryl sulfate (i.e., sodium dodecyl sulfate). The surfactants can be present in an amount from about 0.001% to about 50% by weight, or from about 0.001% to about 20% by weight.

The pharmaceutical compositions of the invention may further comprise an isotonicity agent, such as sodium chloride, dextrose, boric acid, sodium tartrate or other inorganic or organic solutes.

The nasal pharmaceutical compositions of the invention can optionally be used in combination with a pH adjusting agent. Exemplary pH adjusting agents include sulfuric acid, sodium hydroxide, hydrochloric acid, and the like.

To extend shelf life, preservatives can be added to the nasally administrable compositions. Suitable preservatives that can be used include benzyl alcohol, parabens, thimerosal, chlorobutanol, benzalkonium chloride, or mixtures of two or more thereof. Preferably benzalkonium chloride is used. Typically, the preservative will be present in a concentration of up to about 2% by weight. The exact concentration of the preservative, however, will vary depending upon the intended use and can be easily ascertained by one skilled in the art.

Other ingredients which extend shelf life can be added such as for example, antioxidants. Some examples of antioxidants include sodium metabisulfite, potassium metabisulfite, ascorbyl palmitate and the like. Typically, the antioxidant will be present in the compositions in a concentration of from about 0.001% up to about 5% by weight of the total composition.

The nasal delivery systems can be made following the processes described in, for example, U.S. Pat. Nos. 6,451,848, 6,436,950, and 5,874,450, and WO 00/00199, the disclosures of which are incorporated by reference herein in their entirety.

### EXAMPLES

The following examples are for purposes of illustration only and are non intended to limit the spirit or scope of the claims.

### Example 1

The role of dopamine in cognition, emotion and motor performance has been studied. It was reported that there was a link between age related nigrostriatal dopaminergic decline and age-related decline of cognitive function. Erixon-Lindrotha et al, Psychiatry Research: Neuroimaging, 138:1-12 (2005). It was also reported that pharmacological manipulation of dopamine systems could modify cognitive performance in humans. Luciana et al, Cereb. Cortex., 8:218-226 (1998); Kimberg et al, Neuropsychologia, 41:1020-1027 (2003). Facilitation of dopaminergic activity could improve cognitive function in human.

Acetylcholinesterase inhibitors are often utilized for the treatment of dementia. It was reported that donepezil can increase the release of dopamine in the brain, and it was also reported that coadministration with the MAO-B inhibitor selegiline enhanced learning ability. Giacobini et al, Neuropharmacology, 35:205-211 (1996); Takahata et al, Eur. J. Pharmacol., 518:140-144 (2005). It was reported that other drugs for Alzheimer's disease (e.g., memantine) have some effect on dopamine turnover and are effective in combination with donepezil. Bubser et al, Eur. J. Pharmacol., 229:75-82 (1992); Tariot et al, JAMA, 291:317-324 (2004). These results indicated that improvement of cognitive function by acetylcholinesterase inhibitors can be augmented by compounds having positive effects on dopaminergic activity.

C57BL/6 mice (SLC, Shizuoka, Japan) were used for the experiment. 3-(2-cyanophenyl)-5-(2-pyridyl)-1-phenyl-1,2-dihydropyridin-2-one and donepezil were suspended or dissolved in a 0.5% methyl cellulose solution. The two drugs were orally administered by gavage 1 hour before sampling. At the time the mice were sacrificed, both striata were rapidly dissected and were frozen by liquid nitrogen. The samples were subsequently sonicated in 0.5 ml of chilled 0.5 M perchloric acid containing 0.1 M EDTA, and centrifuged at 13000 rpm for 15 min at 4° C. Then monoamine levels were measured by HPLC with electrochemical detection. Supernatant aliquots of 201 were separated over an Eicompak SC5-ODS column (3×150 mm; Eicom, Japan) with mobile phase of 17% methanol, 83% 0.1M acetate/citrate buffer (pH 3.5) containing 190 mg/ml sodium 1-octanesulfonate and 50 mg/ml EDTA, and a flow rate of 0.65 ml/min. Peaks of dopamine, DOPAC, HVA, 5-HT, and 5-HIAA were quantified by area integration with PowerChrom (eDAQ, Australia), and concentrations were expressed as nanograms per milligram of striatal tissue.

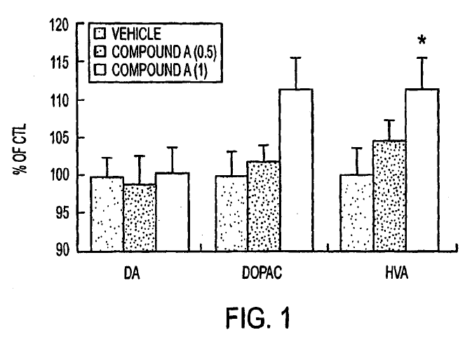

As shown in FIG. 1, 3-(2-cyanophenyl)-5-(2-pyridyl)-1-phenyl-1,2-dihydropyridin-2-one (i.e., Compound A) elevated the content of HVA, which is a metabolite of dopamine in striatum at 1 mg/kg.

### Example 2

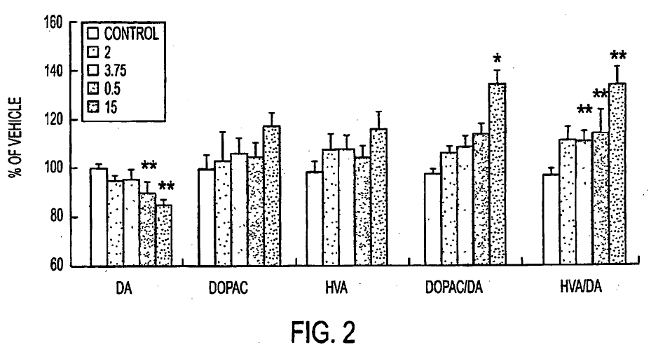

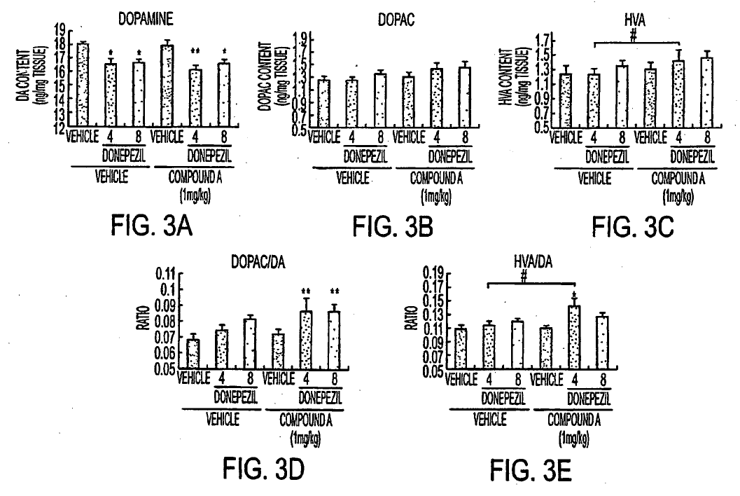

Donepezil reduced the content of dopamine significantly but elevation of dopamine metabolite did not reach significance. FIG. 2 shows that DPAC/DA and HVA/DA, which also indicate increased dopamine turnover, increased significantly. Behavioral symptoms of donepezil were observed in the experiment. Donepezil treated mice showed peripheral side effect at 7.5 mg/kg and higher. These results indicate that both 3-(2-cyanophenyl)-5-(2-pyridyl)-1-phenyl-1,2-dihydropyridin-2-one monotherapy and donepezil monotherapy modulated dopamine turnover but neither reached significance, suggesting a modest effect on dopamine turnover. FIG. 3, however, shows that the combination of 3-(2-cyanophenyl)-5-(2-pyridyl)-1-phenyl-1,2-dihydropyridin-2-one and donepezil unexpectedly facilitated superior dopamine turnover in mice compared to monotherapy with the individual compounds. This experiment demonstrates that the combination therapy of 3-(2-cyanophenyl)-5-(2-pyridyl)-1-phenyl-1,2-dihydropyridin-2-one and donepezil (when compared to monotherapy with either 3-(2-cyanophenyl)-5-(2-pyridyl)-1-phenyl-1,2-dihydropyridin-2-one or donepezil) can be used to treat cognitive dysfunctions, dementia, and neurodegenerative diseases, such as Alzheimer's disease.

### Example 3

Example 5 shows the neuroprotective effects of 3-(2-cyanophenyl)-5-(2-pyridyl)-1-phenyl-1,2-dihydropyridin-2-one and donepezil. A combination of 3-(2-cyanophenyl)-5-(2-pyridyl)-1-phenyl-1,2-dihydropyridin-2-one and donepezil show clear neuroprotective effects against excitotoxicity; however, the profile of both drugs is different. Therefore, complementary approaches to maximize the neuroprotective effects by the combination of drugs may be required to halt progression of neurodegenerative diseases including Alzheimer's disease, Parkinson's disease, Huntington's disease, amyotrophic lateral sclerosis and the like.

It has been described that neurodegenerative diseases exhibit excitotoxicity in the process of neurodegeneration. Rego et al, Neurochemical Research, 28:1563-1574 (2003). 3-(2-cyanophenyl)-5-(2-pyridyl)-1-phenyl-1,2-dihydropyridin-2-one is AMPA antagonist. AMPA antagonists have an effect on excitotoxic conditions induced by kainate and metabolic stress. Ohno et al, JPET, 306:66-72 (2003); Matsuoka et al, Neuroreport, 6:2205-2208 (1995). On the other hand, it is known that AMPA antagonists show no and limited protective effects against NMDA-induced toxicity. Kristensen, Brain Res., 917:21-44 (2001).

Donepezil, commercially available as ARICEPT® (Eisai, Inc., Teaneck, N.J.) is prescribed for Alzheimer's disease. Donepezil improves cognition in patients with dementia, which is caused by acetylcholinesterase inhibition. Donepezil has exhibits neuroprotective effects against excitotoxicity induced by NMDA, but not by kainate. Akasofu et al, Eur. J. Pharmacol., 530:215-222 (2006). The neuroprotective effects of donepezil are supported by clinical findings in brain atrophy of patients with Alzheimer's disease. Hashimoto et al, Am J Psychiatry, 162:676-682 (2005). Donepezil could be a disease modifying drug for neurodegenerative diseases; however, the neuroprotective effects of donepezil are insufficient to completely halt the progression of Alzheimer's disease.

Combination therapy is sometimes beneficial but sometimes harmful. Therefore it is very important to confirm the safety and efficacy of the combination. The combination therapy of the two drugs (3-(2-cyanophenyl)-5-(2-pyridyl)-1-phenyl-1,2-dihydropyridin-2-one and donepezil) in cultured neurons enhanced the beneficial effects against NMDA- and kainate-induced neurodegeneration. Neurodegeneration by the drug combination was not observed. Therefore, the combination of drugs can be a favorable option for the treatment of neurodegeneration, which leads to diseases like Alzheimer's disease, Parkinson's disease, Huntington's disease, amyotrophic lateral sclerosis, and the like.

Cortical cell cultures were prepared from fetal rats of the Wistar strain (gestational age of 17-19 days, Charles River Japan, Kanagawa, Japan). The cortex was dissected and kept in ice-cold Neurobasal medium* ((Neurobasal medium (Gibco BRL) containing 2% B27 supplement (Gibco BRL), 0.5 mM L-glutamine (Gibco BRL), 2% Antibiotic-Antimycotic liquid (Gibco BRL), 25 μM 2-mercaptoethanol)) and then incubated at 37° C. for 30 min in Ca²⁺/Mg²⁺-free HBSS containing 0.25% trypsin (Gibco BRL) and 0.2 mg/ml deoxyribonuclease (DNase) (Sigma, St. Louis, Mo., USA). The cortical tissues were dissociated to single cells by gentle trituration. The cell suspension was mixed with Neurobasal medium with 10% heat-inactivated fetal calf serum (Gibco BRL). The cell suspension was centrifuged at 310×g for 3 min and the resulting pellets were resuspended in the medium described above.

Cells were plated at an initial cell density of 2.0×10⁵ cells/cm² in poly-L-lysine-coated well plates. The cells were cultured in a CO₂ incubator 5% (v/v), at 37° C. in Neurobasal medium containing 2% B27 supplement for 7 days.

The cultures were exposed to 200 μM NMDA (Sigma) or 200 μM kainite in neurobasal medium containing 2% B27 supplement (-AO) for 24 h at 37° C. The cells were treated with donepezil and 3-(2-cyanophenyl)-5-(2-pyridyl)-1-phenyl-1,2-dihydropyridin-2-one for 24 hours before and during the exposure to these agonists.

LDH efflux into the culture medium was measured after NMDA or kainate insult. Remaining LDH in the cells was measured after lysing the cells with 0.1 M phosphate buffer (0.1 M Na₂HPO₄ and 0.1 M KH₂PO₄: pH 7.4) containing 0.5% Triton X-100. LDH release as an indicator of neuronal injury was presented as the percentage leakage of LDH into the culture medium, with respect to the total LDH activity per culture (LDH efflux/total LDH measured). The decrease of NADH was spectrophotometrically monitored at 340 nm as a measure of LDH activity.

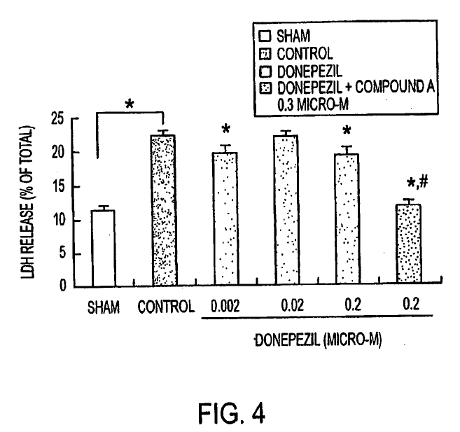

In kainate-induced neuronal toxicity, donepezil showed modest effect on neuronal survival. FIG. 4 shows that the co-administration of donepezil and 3-(2-cyanophenyl)-5-(2-pyridyl)-1-phenyl-1,2-dihydropyridin-2-one significantly and unexpectedly enhanced neuronal survival compared to donepezil alone.

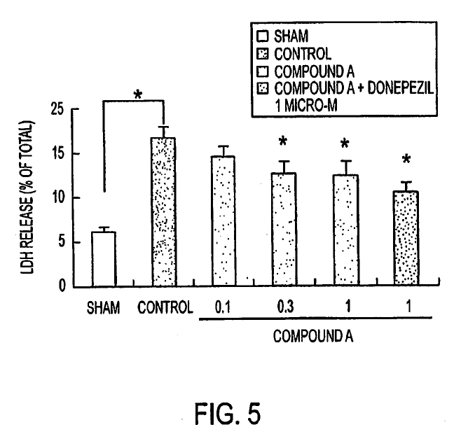

In the NMDA experiment, 3-(2-cyanophenyl)-5-(2-pyridyl)-1-phenyl-1,2-dihydropyridin-2-one showed effects and reached a plateau from 0.3 mM. As shown in FIG. 5, the co-administration of 3-(2-cyanophenyl)-5-(2-pyridyl)-1-phenyl-1,2-dihydropyridin-2-one and donepezil unexpectedly and significantly enhanced neuronal survival.

### Example 4

Organotypic hippocampal slice cultures were prepared using the basic method of Pringle et al, Brain Research, 755:36-46 (1997) modified as follows. Wistar rat pups (8-11 days old) were decapitated and the hippocampus rapidly dissected into ice-cold Gey's balanced salt solution supplemented with 4.5 mg/ml glucose. Transverse sections (400 μm) were cut on a McIlwain tissue chopper and placed back into ice-cold Gey's balanced salt solution. Slices were separated and plated onto Millicell CM culture inserts (4 per well) and maintained at 37° C./5% CO₂ for 14 days. Maintenance medium consisted of 25% heat-inactivated bone serum, 25% Hank's balanced salt solution (HBSS) and 50% minimum essential medium with added Earle's salts (MEM) supplemented with 1 mM glutamine and 4.5 mg/ml glucose. Medium was changed every 3-4 days.

Cultures were transferred to serum free medium (SFM —75% MEM, 25% HBSS) supplemented with 1 mM glutamine and 4.5 mg/ml glucose) containing 5 g/1 ml of the fluorescent exclusion dye propidium iodide (PI), and allowed to equilibrate for 60 minutes prior to imaging. PI fluorescence was detected using a Leica DMIL inverted microscope fitted with a rhodamine filter set. Any cultures in which PI fluorescence was detected at this stage were excluded from further study. 3-(2-cyanophenyl)-5-(2-pyridyl)-1-phenyl-1,2-dihydropyridin-2-one and/or donepezil HCl was added to the SFM and cultures returned to the incubator for 24 hours. After this time, cultures were exposed to a neurotoxic insult (see below for protocols) in the presence of PI and 3-(2-cyanophenyl)-5-(2-pyridyl)-1-phenyl-1,2-dihydropyridin-2-one and/or donepezil HCl before being returned to SFM containing 3-(2-cyanophenyl)-5-(2-pyridyl)-1-phenyl-1,2-dihydropyridin-2-one and/or donepezil HCl. Neuronal damage was assessed by PI fluorescence imaging 24 hours after the initiation of the neurotoxic insult. Neurotoxicity protocols are outlined below.

(1) AMPA: Cultures were transferred to fresh SFM (+PI) containing 30 μM S-AMPA for 3 hours under normal culture conditions. At the end of this period, cultures were returned to fresh SFM for 21 hours prior to determination of neuronal death by PI imaging. GYKI52466 (30 μM) was used as a positive, neuroprotective control.

(2) Oxygen-Glucose Deprivation (OGD): Experimental ischaemia was performed by exposing cultures to oxygen-glucose deprivation (OGD) as described previously. Pringle et al, Brain Research, 755:36-46 (1997); Pringle et al, Stroke, 27:2124-2130 (1996); Morrison et al, Br. J. Pharmacol., 137:1255-1268 (2002). OGD was induced by transferring cultures to glucose-free SFM (+PI) which had been saturated with 95% N₂/5% CO₂. Culture plates (without lids) were then sealed into an airtight chamber in which the atmosphere was saturated with 95% N₂/5% CO₂ by continuously blowing through gas at 10 L/min for ten minutes before being sealed and placed in the incubator for 50 mins (total time of OGD was therefore 60 mins). At the end of the period of OGD cultures were returned to normoxic SFM containing PI and placed back in the incubator for 23 hours.

(3) Hypoxia: Hypoxia was performed by exposing cultures to oxygen-deprivation as described previously. Pringle et al, Brain Research, 755:36-46 (1997); Pringle et al, Stroke, 27:2124-2130 (1996). Hypoxia was induced by transferring cultures to SFM (+PI) which had been saturated with 95% N₂/5% CO₂. Culture plates (without lids) were then sealed into an airtight chamber in which the atmosphere was saturated with 95% N₂/5% CO₂ by continuously blowing through gas at 10 L/min for ten minutes before being sealed and placed in the incubator for 170 mins (total time of hypoxia was therefore 180 mins). At the end of the period of hypoxia, cultures were returned to normoxic SFM (+PI) and placed back in the incubator for 21 hours. The freeradical scavenger MnTBAP (100 μM) was used as a positive neuroprotective control.

(4) NMDA: Cultures were transferred to fresh SFM (+PI) containing 10 μM NMDA for 3 hours under normal culture conditions. At the end of this period, cultures were returned to fresh SFM for 21 hours-prior to determination of neuronal death by PI imaging. MK801 (10 μM) was used as a positive, neuroprotective control.

(5) Free radical-mediated toxicity (Duroquinone): Duroquinone produces superoxide-mediated neurotoxicity producing a selective neuronal lesion in the CA1 region. Wilde et al, J. Neurochem., 69(2):883-886 (1997). Cultures were transferred to fresh SFM (+PI) containing 100 μM duroquinone for 3 hours under normal culture conditions. At the end of this period, cultures were returned to fresh SFM for 21 hours prior to determination of neuronal death by PI imaging. MnTBAP (100 μM) was used as a positive neuroprotective control.

Neuronal damage was assessed using ImageJ software running on a PC. Images were captured using a monochrome CCD camera and saved for otlline analysis. Light transmission images were captured prior to the addition of drugs, and PI fluorescence images recorded at the end of the 24-hour recovery period. The area of the CA 1 region was determined from the transmission image. The area of PI fluorescence in the CA1 was measured using the threshold function on ImageJ, and neuronal damage expressed as the percentage of the CA1 in which PI fluorescence was detected above background. Morrison et al, Br. J. Pharmacol., 137:1255-1268 (2002).

MnTBAP and MK-801 were dissolved in distilled water to 10 mM. NMDA was dissolved in distilled water to 100 mM, and AMPA was dissolved in distilled water to 3 mM. GYKI52466 was dissolved in DMSO to 3 mM. Stock solutions (1 mM) of donepezil were prepared fresh each day in distilled water, and then serially diluted into SFM. Stock solutions of 3-(2-cyanophenyl)-5-(2-pyridyl)-1-phenyl-1,2-dihydropyridin-2-one (1 mM) were prepared fresh each day in DMSO and further diluted into SFM.

Materials were obtained from the following suppliers: tissue culture plates —Nunc (Fisher Scientific, Loughborough, UK); all other tissue culture plastics were purchased from—Bibby Sterilin (Stone, UK); culture media—Invitrogen (Paisley, UK); PI—Molecular Probes (Leiden, Holland); S-AMPA, GYKI52466, NMDA, MK801 and MnTBAP —Tocris (Bristol, UK); 3-(2-cyanophenyl)-5-(2-pyridyl)-1-phenyl-1,2-dihydropyridin-2-one, donepezil hydrochloride (Eisai Co. Ltd).

Data analysis was performed using GraphPad Prism. Data were initially assessed for normality after which statistical significance was determined using a one way analysis of variance followed by a Dunnett's post-hoc test. All data is expressed as mean±s.e.m. Raw data and analysis of individual datesets are not provided herewith.

The EC50 for 3-(2-cyanophenyl)-5-(2-pyridyl)-1-phenyl-1,2-dihydropyridin-2-one in AMPA toxicity was determined. No PI fluorescence was observed in slice cultures maintained in SFM for up to 49 hours (sham controls). In contrast, significant neurodegeneration was observed in the CA1 region of cultures exposed to 30 μM AMPA for 3 hours (65.1±1.7% damage in CA1, n=104, p<0.001 vs sham controls). AMPA-mediated neurodegeneration was significantly attenuated by the non-competitive AMPA receptor antagonist GYKI52466, which reduced PI fluorescence to 14.3±2.3% damage (n=60, p<0.001 vs AMPA). AMPA-induced neurodegeneration was reduced in a concentration-dependent manna by 3-(2-cyanophenyl)-5-(2-pyridyl)-1-phenyl-1,2-dihydropyridin-2-one. At 0.1 μM 3-(2-cyanophenyl)-5-(2-pyridyl)-1-phenyl-1,2-dihydropyridin-2-one, no neuroprotective efficacy was observed (63.9±2.6% damage, n=36, p=NS vs AMPA). When the concentration of 3-(2-cyanophenyl)-5-(2-pyridyl)-1-phenyl-1,2-dihydropyridin-2-one was increased to 0.3 μM, significant neuroprotection was observed, with damage reduced to 50.4±2.5% (n=60, p<0.01 vs AMPA). At the highest concentration of 3-(2-cyanophenyl)-5-(2-pyridyl)-1-phenyl-1,2-dihydropyridin-2-one (3 μM), neuroprotection was almost complete (0.1±0.1% damage, n=36, p<0.001 vs AMPA). Based on this series of experiments, the EC50 for 3-(2-cyanophenyl)-5-(2-pyridyl)-1-phenyl-1,2-dihydropyridin-2-one in this model of AMPA toxicity was determined to be 0.5 μM.

The effect of donepezil on AMPA-mediated toxicity was determined. Initially, a series of cultures (24 per group) was exposed to either SFM or donepezil (0.1 to 10 μM) for 48 hours (solutions were changed for ones containing flesh donepezil after 24 hours) to assess potential toxicity of the compound. No PI fluorescence was observed in any culture in this experiment indicating that the compound was nontoxic in this paradigm (data not shown).

A further set of preliminary experiments was performed in order to determine the effect of increasing the pre-incubation time (4 to 48 hours) of donepezil prior to exposure of cultures to AMPA. As previously, no PI fluorescence was observed in cultures maintained in SFM for the duration of the experiment, but significant toxicity was observed in CA1 following exposure to AMPA (46.5±5.0% damage, n=24). This was significantly attenuated by 30 μM GYKI52466 (21.7±2.7% damage, n=24, p<0.001 vs AMPA). No effect of donepezil was observed in the longer pre-incubation times. PI 55.51±2.6% of CA1 (24 hours pre-incubation, n=24, p=NS), respectively. However, when the pre-incubation time was decreased to 4 hours, a small but significant increase in AMPA-mediated toxicity was observed, with PI fluorescence being observed in 57.4±2.3% of CA1 (n=24, p<0.05 vs AMPA).

No biologically significant effect was observed by altering the pre-incubation period; therefore, a standard pre-incubation period of 24 hours was chosen for the remainder of the experiments based on published evidence. Akasofu et al, Eur. J. Pharmacol., 472(1-2):57-63 (2003); Takada et al, Pharmacol. Exp. Ther., 306(2):772-777 (2003).

In a further series of experiments, exposure of cultures to 30 μM AMPA resulted in PI fluorescence occurring in 62.4±2.4% of the CA1 region (n=72). This was significantly attenuated by 0.5 μM 3-(2-cyanophenyl)-5-(2-pyridyl)-1-phenyl-1,2-dihydropyridin-2-one, reducing the area of PI fluorescence to 33.6±4.0% of CA1 (n=24, p<0.001 vs AMPA). No neuroprotective effect of donepezil was observed at any of the concentrations tested (0.1 to 10 μM). As observed previously (FIGS. 8 and 9), we observed a small, but non-significant increase in AMPA-mediated toxicity in the presence of all three concentrations of donepezil (0.1 μM: 69.5±2.4% of CA1; 1 μM: 70.7±2.4% of CA1; 10 μM: 70.9%±2.8% of CA1; all groups n=36, p=NS vs AMPA). Although 0.5 μM 3-(2-cyanophenyl)-5-(2-pyridyl)-1-phenyl-1,2-dihydropyridin-2-one was significantly neuroprotective when present alone, the efficacy of 3-(2-cyanophenyl)-5-(2-pyridyl)-1-phenyl-1,2-dihydropyridin-2-one was lost when the compound was co-incubated with donepezil. This loss of efficacy was statistically significant at all three concentrations of donepezil (0.1 μM donepezil+-(2-cyanophenyl)-5-(2-pyridyl)-1-phenyl-1,2-dihydropyridin-2-one: 70.2±1.6% of CA1; 1μM: 62.6±3.6% of CA1; 10 μM: 68.7%±2.4% of CA 1; all groups n=36, p<0.001 vs AMPA).

Experimental ischaemia was induced by exposing cultures to 60 minutes oxygen-glucose deprivation (OGD). An initial set of experiments was conducted to determine the neuroprotective efficacy of 3-(2-cyanophenyl)-5-(2-pyridyl)-1-phenyl-1,2-dihydropyridin-2-one against OGD-induced neurotoxicity. Exposure of cultures to 60 minutes OGD produced extensive PI fluorescence in the CA1 region (50.6±5.2% damage, n=24) and this was prevented by the fee-radical scavenger MnTBAP (100 μM) (0.1±0.0% damage, n=24, p<0.001 vs OGD). No significant neuroprotection was observed in cultures treated with the non-competitive AMPA receptor antagonist GYKI52466 (30 μM) (36.9±3.6%, n=24, p=NS). In order to assess the EC50 for 3-(2-cyanophenyl)-5-(2-pyridyl)-1-phenyl-1,2-dihydropyridin-2-one, a six-point concentration-response curve was generated. Surprisingly, in cultures treated with the lowest concentration of 3-(2-cyanophenyl)-5-(2-pyridyl)-1-phenyl-1,2-dihydropyridin-2-one (0.1 μM), a significant increase in PI fluorescence was observed (64.4±3.6%, n=24, p<0.05 vs OGD). There was no significant effect of 3-(2-cyanophenyl)-5-(2-pyridyl)-1-phenyl-1,2-dihydropyridin-2-one at any other concentration assessed (up to 3 μM).

As a separate EC50 could not be established in this model, further experiments were performed using 0.5 μM 3-(2-cyanophenyl)-5-(2-pyridyl)-1-phenyl-1,2-dihydropyridin-2-one, the EC50 for blockade of AMPA toxicity.

In a second series of experiments, the effects of donepezil were investigated on OGD induced neurotoxicity, and how this was affected by 3-(2-cyanophenyl)-5-(2-pyridyl)-1-phenyl-1,2-dihydropyridin-2-one. In cultures exposed to OGD alone, PI fluorescence occurred in 44.8±3.3% of the CA1 region (n=48). As previously, this was fully attenuated by 100 μM MnTBAP (0.6±0.2% damage, n=48, p<0.001 vs OGD), but not by 0.5 μM 3-(2-cyanophenyl)-5-(2-pyridyl)-1-phenyl-1,2-dihydropyridin-2-one (55.3±3.1% damage, n=32, p=NS). Pretreatment with donepezil (0.1 to 10 μM) had no effect on neuronal damage (0.1 μM: 49.4±2.7% damage; 1 μM: 54.1±4.7% damage; 10 μM: 45.3±3.3%; all n=48, p=NS vs OGD). In cultures exposed to OGD following co-application of 1 μM donepezil and 0.5 μM 3-(2-cyanophenyl)-5-(2-pyridyl)-1-phenyl-1,2-dihydropyridin-2-one there was a small, but significant increase in PI fluorescence (64.3±4.2% damage, n=48, p<0.05 vs OGD). No effect was observed at higher (10 μM) or lower (0.1 μM) concentrations of donepezil, suggesting that this effect may not be biologically relevant.

Duroquinone-induced injury was determined. Free radical-induced injury was produced by exposure of cultures to 100 μM duroquinone for 3 hours followed by a 21 hour recovery period. Cultures exposed to 100 μM duroquinone for 3 hours developed neurodegeneration in the CA1 region, resulting in extensive PI fluorescence (51.0±3.4% damage, n−72). As expected, duroquinone-induced injury was fully abolished by, the free radical scavenger MnTBAP (100 μM) (0.1±0.0% damage, n=24, p<0.001 vs duroquinone). 3-(2-cyanophenyl)-5-(2-pyridyl)-1-phenyl-1,2-dihydropyridin-2-one (0.5 μM) was not neuroprotective in this model (69.1±4.0% damage, n=24, p—NS). Donepezil did not reduce duroquinone-induced neuronal damage (0.1 μM: 56.4±4.4% damage; 1 μM: 53.2±4.9% damage; 10 μM: 56.6±4.4% damage, all n=36, p=NS). Similarly, co-incubation with 3-(2-cyanophenyl)-5-(2-pyridyl)-1-phenyl-1,2-dihydropyridin-2-one and donepezil did not affect neuronal damage.

NMDA-induced neurotoxicity was investigated. Exposure of cultures to 10 μM NMDA for 3 hours produced neurodegeneration in the CA1 region as determined by a significant increase in PI fluorescence (35.1±2.2% damage, n=72), and this was fully attenuated by the non-competitive NMDA receptor antagonist MK-801 (10 μM) (0.0±0.0% damage, n=36, p<0.001 vs NMDA). In contrast, NMDA toxicity was unaffected by 3-(2-cyanophenyl)-5-(2-pyridyl)-1-phenyl-1,2-dihydropyridin-2-one (0.5 μM) (29.1±2.9% damage, n=36, p=NS). Donepezil was significantly neuroprotective at all three concentrations tested in a concentration-dependent manner (0.1 μM: 23.0±2.4% damage, p<0.01 vs NMDA; 1 μM: 25.2±2.3% damage, p<0.05 vs NMDA; 10 μM: 14.5±2.2% damage p<0.001 vs NMDA; all groups n=36). The neuroprotective efficacy of low concentrations of donepezil was attenuated by inclusion of 0.5 μM 3-(2-cyanophenyl)-5-(2-pyridyl)-1-phenyl-1,2-dihydropyridin-2-one (0.1 μM+3-(2-cyanophenyl)-5-(2-pyridyl)-1-phenyl-1,2-dihydropyridin-2-one: 27.9±2.6% damage; 1 μM+3-(2-cyanophenyl)-5-(2-pyridyl)-1-phenyl-1,2-dihydropyridin-2-one: 29.5±3.0% damage; both groups n=36). 10 μM donepezil remained neuroprotective in the presence of 3-(2-cyanophenyl)-5-(2-pyridyl)-1-phenyl-1,2-dihydropyridin-2-one (24.9±2.4% damage, n=36, p<0.05 vs NMDA).

Hypoxia-induced neurodegeneration was examined. Hypoxia-mediated neuronal damage was produced by exposing cultures to oxygen deprivation for a period of 3 hours followed by a 21 hour recovery period. 3 hours hypoxia resulted in a lesion within CA1, as defined by PI fluorescence (45.1±3.5% damage, n=96). This was prevented by the free-radical scavenger MnTBAP (100 μM) (0.2±0.1% damage, n−48, p<0.001 vs hypoxia). Donepezil (0.1 to 10 μM) was highly neuroprotective in this model producing a significant reduction in neuronal damage at the 0.1 μM (31.7±4.6% damage, n=48, p<0.05 vs hypoxia). Neuroprotective efficacy was increased when the concentration of donepezil was increased to 1 μM (14.9±3.2% damage, n=48, p<0.001 vs hypoxia). No further increase in efficacy was observed when the concentration of donepezil was increased to 10 μM (12.9±2.9% damage, n=48, p<0.001 vs hypoxia). A concentration-dependent neuroprotective effect of 3-(2-cyanophenyl)-5-(2-pyridyl)-1-phenyl-1,2-dihydropyridin-2-one was also observed, with significant efficacy being observed at 0.1 μM (24.8±6.3% damage, n=24, p<0.001 vs hypoxia). At the highest concentration tested (5 μM 3-(2-cyanophenyl)-5-(2-pyridyl)-1-phenyl-1,2-dihydropyridin-2-one), the compound was almost as effective as MmTBAP, reducing damage to 3.9±1.8% (n=24, p<0.001 vs hypoxia).

A second set of experiments was performed to investigate whether the neuroprotective effects of donepezil and 3-(2-cyanophenyl)-5-(2-pyridyl)-1-phenyl-1,2-dihydropyridin-2-one were synergistic or additive. In order to test the hypothesis, the lowest effective concentrations (i.e., 0.1 μM) of each of the two compounds was used). Hypoxia alone resulted in 36.4±3.9% damage (n=48), and this was prevented by MnTBAP (1100 M) (0.0±0.0%, n=24, p<0.001 vs hypoxia). Neither donepezil (0.1 μM: 30.4±3.9% damage, n=48) nor 3-(2-cyanophenyl)-5-(2-pyridyl)-1-phenyl-1,2-dihydropyridin-2-one (0.1 μM: 36.7±4.2% damage, n=48, p=NS) were neuroprotective in this series of experiments. Similarly, no reduction in PI fluorescence was observed in culture incubated with both compounds together (33.8±4.1% damage, n=48, p=NS).

It is likely that 0.1 μM lies very close to the threshold concentration for neuroprotective efficacy for donepezil and 3-(2-cyanophenyl)-5-(2-pyridyl)-1-phenyl-1,2-dihydropyridin-2-one. The experiment using 0.5 μM donepezil and 0.5 μM 3-(2-cyanophenyl)-5-(2-pyridyl)-1-phenyl-1,2-dihydropyridin-2-one was repeated. In this experiment, exposure of cultures to hypoxia resulted in 36.9±4.4% damage (n=48) and this was blocked by 100 μM MnTBAP (0.8±0.4% damage, n=24, p<0.001 vs hypoxia). Donepezil (0.5 μM) produced a significant decrease in neuronal damage (25.3±3.6% damage, n=48, p<0.05 vs hypoxia), an effect mirrored by 3-(2-cyanophenyl)-5-(2-pyridyl)-1-phenyl-1,2-dihydropyridin-2-one (0.5 μM) (27.1±3.5% damage, n=48, p<0.05 vs hypoxia). A similar level of neuroprotection was observed when donepezil and 3-(2-cyanophenyl)-5-(2-pyridyl)-1-phenyl-1,2-dihydropyridin-2-one were used in combination (24.7±3.5% damage, n=48, p<0.05 vs hypoxia).

Each of the patents, patent applications, and publications cited herein are incorporated by reference herein in their entirety.

It will be apparent to one skilled in the art that various modifications can be made to the invention without departing from the spirit or scope of the appended claims.

What is claimed is:

1. A method for treating Alzheimer's disease in a patient in need thereof comprising administering a therapeutically effective amount of: (i) 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof; and (ii) donepezil or a pharmaceutically acceptable salt thereof.

2. The method of claim 1, wherein donepezil or the pharmaceutically acceptable salt thereof, and 3-(2-cyanophenyl)-5-(2-pyridyl)-1-phenyl-1,2-dihydropyridin-2-one, the pharmaceutically acceptable salt thereof, the hydrate thereof, or the hydrate of the pharmaceutically acceptable salt thereof are administered separately to the patient.

3. The method of claim 1, wherein donepezil or the pharmaceutically acceptable salt thereof, and 3-(2-cyanophenyl)-5-(2-pyridyl)-1-phenyl-1,2-dihydropyridin-2-one, the pharmaceutically acceptable salt thereof, the hydrate thereof, or the hydrate of the pharmaceutically acceptable salt thereof are administered to the patient in the form of a pharmaceutical composition.

4. The method of claim 1, wherein the therapeutically effective amount of 3-(2-cyanophenyl)-5-(2-pyridyl)-1-phenyl-1,2-dihydropyridin-2-one is 30 μg to 500 milligrams.

5. The method of claim 1, wherein the therapeutically effective amount of donepezil is 1 mg to 50 mg.

6. A method for treating dementia or one or more cognitive impairments in a patient in need thereof comprising administering a therapeutically effective amount of: (i) 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof; and (ii) donepezil or a pharmaceutically acceptable salt thereof.

7. The method of claim 6, wherein donepezil or the pharmaceutically acceptable salt thereof, and 3-(2-cyanophenyl)-5-(2-pyridyl)-1-phenyl-1,2-dihydropyridin-2-one, the pharmaceutically acceptable salt thereof, the hydrate thereof, or the hydrate of the pharmaceutically acceptable salt thereof are administered separately to the patient.

8. The method of claim 6, wherein donepezil or the pharmaceutically acceptable salt thereof, and 3-(2-cyanophenyl)-5-(2-pyridyl)-1-phenyl-1,2-dihydropyridin-2-one, the pharmaceutically acceptable salt thereof, the hydrate thereof, or the hydrate of the pharmaceutically acceptable salt thereof are administered to the patient in the form of a pharmaceutical composition.

9. The method of claim 6, wherein the therapeutically effective amount of 3-(2-cyanophenyl)-5-(2-pyridyl)-1-phenyl-1,2-dihydropyridin-2-one is 30 μg to 500 milligrams.

10. The method of claim 6, wherein the therapeutically effective amount of donepezil is 1 mg to 50 mg.

11. The method of claim 6, wherein the dementia is caused by Alzheimer's disease, Parkinson's disease, or Huntington's disease.

12. A pharmaceutical composition comprising a therapeutically effective amount of: (i) donepezil or a pharmaceutically acceptable salt thereof; and (ii) 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

13. The composition of claim 12, wherein donepezil is present in an amount from 1 milligram to 50 milligrams.

14. The composition of claim 12, wherein the 3-(2-cyanophenyl)-5-(2-pyridyl)-1-phenyl-1,2-dihydropyridin-2-one is present in an amount from 30 micrograms to 1 gram.

15. A method for treating dementia, one or more cognitive impairments, or a neurodegenerative disease in a patient in need thereof comprising administering a therapeutically effective amount of: (i) a compound of Formula (III), a pharmaceutically acceptable salt thereof, a hydrate thereof, or a hydrate of a pharmaceutically acceptable salt thereof; and (ii) a compound of Formula (VI) or a pharmaceutically acceptable salt thereof;
wherein the compound of Formula (III) is: 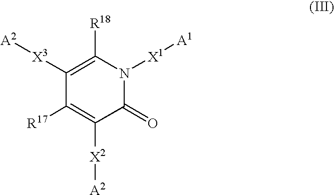
wherein
X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O), —N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);
R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;
m, n, p and q are each independently an integer of 0, 1 or 2;
A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and
R¹⁷ and R¹⁸ are each independently hydrogen, halogen, or C₁₋₆ alkyl; and wherein the compound of Formula (VI) is: 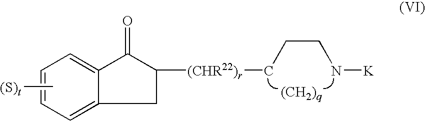
wherein
r is an integer of 1 to 10;
each R²² is independently hydrogen or methyl;
K is a phenalkyl or a phenalkyl having a substituent on the phenyl;
each S is independently hydrogen, C₁₋₆ lower alkyl, or C₁₋₆ lower alkoxy;
t is an integer of 1 to 4;
q is an integer of 1 to 3;
with the proviso that (S)ₜ can be methylenedioxy or ethylenedioxy joined to two adjacent carbon atoms of the phenyl.

16. The method of claim 15, wherein the neurodegenerative disease is Alzheimer's disease, Parkinson's disease, Huntington's disease, or amyotrophic lateral sclerosis.

17. The method of claim 15, wherein the dementia is caused by Alzheimer's disease, Parkinson's disease, or Huntington's disease.

18. The method of claim 15, wherein the compound of Formula (VI) is present in an amount from 1 milligram to 50 milligrams.

19. The method of claim 15, wherein the compound of Formula (III) is present in an amount from 30 micrograms to 1 gram.

20. The method of claim 15, wherein the compound of Formula (III), the pharmaceutically acceptable salt thereof, the hydrate thereof, or the hydrate of the pharmaceutically acceptable salt thereof; and the compound of Formula (VI) or the pharmaceutically acceptable salt thereof, are administered separately to the patient.

21. The method of claim 15, wherein the compound of Formula (III), the pharmaceutically acceptable salt thereof, the hydrate thereof, or the hydrate of the pharmaceutically acceptable salt thereof; and the compound of Formula (VI) or the pharmaceutically acceptable salt thereof, are administered to the patient in the form of a pharmaceutical composition.

22. A pharmaceutical composition comprising a therapeutically effective amount of: (i) a compound of Formula (III), a pharmaceutically acceptable salt thereof, a hydrate thereof, or a hydrate of a pharmaceutically acceptable salt thereof; and (ii) a compound of Formula (VI) or a pharmaceutically acceptable salt thereof;
wherein the compound of Formula (III) is: 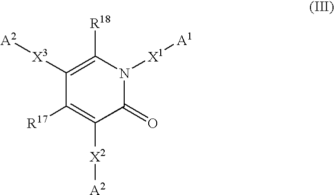
wherein
X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₘ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);
R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;
m, n, p and q are each independently an integer of 0, 1 or 2;
A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and
R¹⁷ and R¹⁸ are each independently hydrogen, halogen, or C₁₋₆ alkyl; and
wherein the compound of Formula (VI) is: 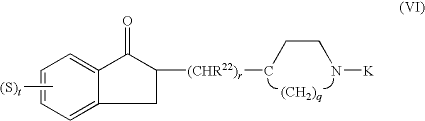
wherein
r is an integer of 1 to 10;
each R²² is independently hydrogen or methyl;
K is a phenalkyl or a phenalkyl having a substituent on the phenyl;
each S is independently hydrogen, C₁₋₆ lower alkyl, or C₁₋₆ lower alkoxy;
t is an integer of 1 to 4;
q is an integer of 1 to 3;
with the proviso that (S)ₜ can be methylenedioxy or ethylenedioxy joined to two adjacent carbon atoms of the phenyl.

23. The composition of claim 22, wherein the compound of Formula (III) is present in an amount of 30 μg to 10 grams.

24. The composition of claim 22, wherein the compound of Formula (VI) is present in an amount from 1 mg to 50 mg.

25. A combination comprising at least one AMPA receptor antagonist and at least one nootropic, in which the active ingredients are present in each case in a free form or in the form of a pharmaceutically acceptable salt, and, optionally, at least one pharmaceutically acceptable carrier; for simultaneous separate or sequential use.

26. The combination of claim 25 which is a combined preparation or a pharmaceutical composition.

27. The combination of claim 25, wherein the nootropic is a cholinesterase inhibitor.

28. The combination of claim 27, wherein the cholinesterase inhibitor is donepezil or a pharmaceutically acceptable salt thereof; rivastigmine or a pharmaceutically acceptable salt thereof; or galantamine or a pharmaceutically acceptable salt thereof.

29. The combination of claim 27, wherein the cholinesterase inhibitor is donepezil or a pharmaceutically acceptable salt thereof

30. The combination of claim 25, wherein the AMPA receptor antagonist is a 1,2-dihydropyridine compound.

31. The combination of claim 30, wherein the 1,2-dihydropyridine compound is 3-(2-cyanophenyl)-5-(2-pyridyl)-1-phenyl-1,2-dihydropyridin-2-one or a pharmaceutically acceptable salt thereof.

32. The combination of claim 25 for simultaneous, separate or sequential use in the treatment of dementia.

33. The combination of claim 25 for simultaneous, separate or sequential use in the treatment of behavioral disturbances observed with dementia.

34. A method for treating dementia or one or more behavioral disturbances observed with dementia in a patient in need thereof comprising administering the combination of claim 25 in a quantity which is jointly therapeutically effective in dementia or behavioral disturbances observed with dementia and in which the compounds can be present in the form of their pharmaceutically acceptable salts.

35. A commercial package comprising the combination of claim 25 with instructions for simultaneous, separate or sequential use thereof in the treatment of dementia or behavioral disturbances observed with dementia.

36. A pharmaceutical composition comprising both an AMPA receptor antagonist and a nootropic in a quantity which is jointly therapeutically effective; and a pharmaceutically acceptable carrier.
